(19) United States
(12) Patent Application Publication
(10) Pub. No.: US 2025/0297262 A1
(43) Pub. Date: Sep. 25, 2025

(54) REGULATION OF TYPE I IFN SIGNALING BY TARGETING A DECOY RECEPTOR

(71) Applicant: THE REGENTS OF THE UNIVERSITY OF COLORADO A BODY CORPORATE, Denver, CO (US)

(72) Inventors: Edward CHUONG, Boulder, CO (US); Giulia PASQUESI, Broomfield, CO (US)

(21) Appl. No.: 18/864,230

(22) PCT Filed: May 9, 2023
(86) PCT No.: PCT/US2023/066767

§ 371 Date: Nov. 8, 2024

Related U.S. Application Data
(60) Provisional application No. 63/339,572, filed on May 9, 2022.

(51) Int. Cl. C12N 15/113; A61K 48/00; C12N 9/22; C12N 15/11

(52) U.S. Cl. CPC C12N 15/1138; A61K 48/005; C12N 9/226; C12N 15/111; C12N 2310/11; C12N 2310/14; C12N 2310/20; C12N 2320/33

(57) ABSTRACT

The present invention includes systems, methods and compositions to modulate the IFN signaling pathway and its downstream effects on the innate immune response by regulating the expression of one or more IFNAR2 isoforms. In a preferred aspect, the modulation of the IFN signaling pathway may be accomplished by modulating the relative ration between IFNAR2 isoforms, IFNAR2-L and IFNAR2-S.

### CROSS REFERENCE TO RELATED APPLICATIONS

This International PCT application claims the benefit of and priority to U.S. Provisional Application No. 63/339,572, filed May 9, 2022. The specification, claims and drawings of which are incorporated herein by reference in their entirety.

### SEQUENCE LISTING

The instant application contains contents of the electronic sequence listing (90245-00791-Sequence-Listing.xml; Size: 3,259,809 bytes; and Date of Creation: May 9, 2023) is herein incorporated by reference in its entirety.

### TECHNICAL FIELD

The present invention relates to systems, methods, and compositions to regulate the type I Interferon (IFN) signaling pathway in a human subject and its downstream effects on the innate immune response.

### BACKGROUND

The IFN signaling pathway controls the innate immune response of human cells, and has been an important therapeutic target for over 30 years. Recombinant IFN is widely used in the treatment of several cancers, autoimmune disorders including multiple sclerosis, and viral infections. Dysregulation of IFN signaling is correlated with severe COVID-19 and administration of IFNs are under clinical trials for SARS-CoV2 infection. Despite the widespread importance of IFN-related therapies, unsolved challenges include off-target toxicity and acquired cellular resistance to IFN signaling. New methods are needed to enable less toxic and more effective control of cellular sensitivity to IFN signaling.

The Interferon Alpha And Beta Receptor Subunit 2 (IFNAR2) cytokine receptor (SEQ ID NO. 1) is a core component of type I IFN signaling. The human IFNAR2 genomic locus contains multiple transcript isoforms, with the canonical full-length protein encoded by the longest isoform (IFNAR2-L) (SEQ ID NO. 4, and 7). An alternative short IFNAR2-S isoform (SEQ ID NO. 2, and 6) shares the first 8 exons with IFNAR2-L but is truncated by an early terminal 9th exon, derived from a primate-specific Alu repeat insertion. The resulting transcript is predicted to produce a truncated receptor that contains intact ligand-binding and transmembrane domains, but lacks the cytoplasmic signaling domain. However, the short IFNAR2-S isoform is assumed to be nonfunctional, and the long IFNAR2-L isoform is widely assumed to be the only source of IFNAR2 protein in human cells.

In contrast to this expectation, as described below, the present inventors have demonstrated through transcriptomic analysis that IFNAR2-S is expressed at higher levels than the long IFNAR2-L isoform in most human tissues. Isoform-specific analysis and functional dissection of IFNAR2-S and IFNAR2-L was conducted which facilitated the discovery that IFNAR2-S functions as a decoy receptor that negatively regulates type I IFN signaling in human cells. Based on this new understanding, the present inventors demonstrate new strategies to modulate IFN responses by modulating expression of IFNAR2 isoforms, for example by overexpression or silencing. As described below, the modulating expression of IFNAR2 isoforms may have significant therapeutic applications.

### SUMMARY OF THE INVENTION

In one aspect, the present invention includes systems, methods and compositions to modulate the IFN signaling pathway in a human subject and its downstream effects on the innate immune response by regulating the expression of one or more IFNAR2 isoforms. In a preferred aspect, the modulation of the IFN signaling pathway may be accomplished by increasing or decreasing the expression of one or more IFNAR2 isoforms, and preferably IFNAR2-L and IFNAR2-S.

In another aspect, the present invention includes systems, methods and compositions to increase or decrease the IFN signaling pathway in a subject, and preferably a human subject though the manipulation of the relative ratio and/or expression levels or one or more IFNAR2 isoforms, and preferably IFNAR2-L and IFNAR2-S. In one preferred embodiment, the present invention may include downregulating the IFN signaling pathway in a subject through increasing the relative expression/population of IFNAR2-S, compared to IFNAR2-L. In another preferred embodiment, the present invention may include upregulating the IFN signaling pathway in a subject through increasing the relative expression/population of IFNAR2-L, compared to IFNAR2-S.

In another aspect, the present invention includes systems, methods and compositions to increase or decrease the IFN mediated innate immune response in a subject, and preferably a human subject though the manipulation of the relative ratio and/or expression levels or one or more IFNAR2 isoforms, and preferably IFNAR2-L and IFNAR2-S. In one preferred embodiment, the present invention may include downregulating the innate immune response in a subject through increasing the relative expression/population of IFNAR2-S, compared to IFNAR2-L. In another preferred embodiment, the present invention may include upregulating the innate immune response in a subject through increasing the relative expression/population of IFNAR2-L, compared to IFNAR2-S.

In another aspect, the present invention includes systems, methods and compositions to modulate the IFN signaling pathway in a human subject and its downstream effects on the innate immune response by downregulating expression of one or more IFNAR2 isoforms. In a preferred embodiment, downregulating expression of one or more IFNAR2 isoforms may include inhibiting expression through a RNA interference directed to one or more IFNAR2 isoforms, and in particular IFNAR2-L and IFNAR2-S. In another preferred aspect, downregulating expression of one or more IFNAR2 isoforms may include knocking out, for example through a directed endonuclease system, such as CRISPR/Cas9, of one or more IFNAR2 isoforms, and in particular IFNAR2-L and IFNAR2-S.

In another aspect, the present invention includes systems, methods and compositions to control cellular sensitivity to interferon signaling and its downstream effects on the innate immune response by manipulating the cellular IFNAR2 isoform ratio. In a preferred aspect, the control cellular sensitivity to interferon signaling and its downstream effects on the innate immune response by manipulating the cellular ratio between IFNAR2-L and IFNAR2-S.

In another aspect, the present invention includes systems, methods and compositions to treat cancer though the manipulation of the relative ratio and/or expression levels or one or more IFNAR2 isoforms, and preferably IFNAR2-L and IFNAR2-S. In one preferred embodiment, the present invention may include downregulating the IFN signaling pathway in a subject having an IFN resistant form of cancer through increasing the relative expression/population of IFNAR2-S, compared to IFNAR2-L. In another preferred embodiment, the present invention may include upregulating the IFN signaling pathway in a subject having an inflammatory auto-immune cancer through increasing the relative expression/population of IFNAR2-L, compared to IFNAR2-S.

In another aspect, the present invention includes systems, methods and compositions to treat infection, and preferably a viral infection, though the manipulation of the relative ratio and/or expression levels or one or more IFNAR2 isoforms, and preferably IFNAR2-L and IFNAR2-S. In another preferred embodiment, the present invention may include treatment of a viral infection by upregulating the innate immune response in a subject through increasing the relative expression/population of IFNAR2-L, compared to IFNAR2-S.

### BRIEF DESCRIPTION OF THE DRAWINGS

FIG. 1 shows a schematic comparison between the canonical IFN signaling pathway and the application of “short” isoform IFNAR2-S that acts as an IFN decoy receptor, and wherein the ratio of short/long isoform levels controls sensitivity to IFN signaling.

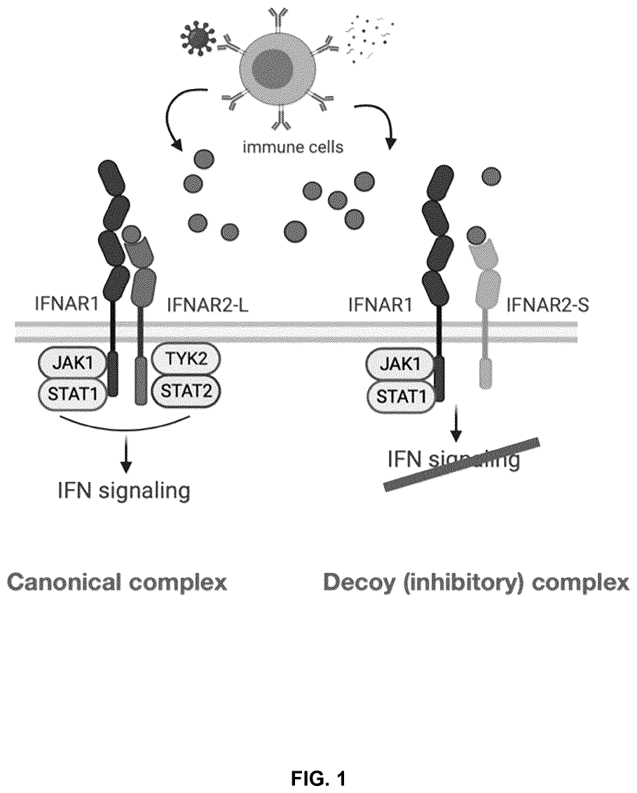

FIG. 2 shows a schematic diagram of a Type I interferon (IFN) response and its relationship to host cellular innate immunity response.

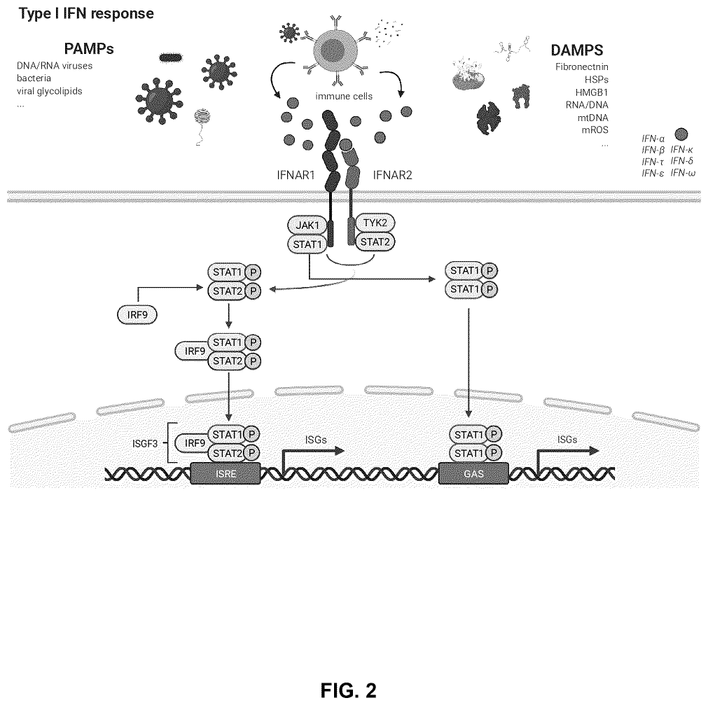

FIG. 3 show exemplary isoforms of IFNAR2, specifically IFNAR2-L, IFNAR2-S, and IFNAR2-sol. These three isoforms are generated by alternative splicing, and can further be designated as: 1) “Long” which describes a full length, canonical functional isoform (Jak1 binding domain); 2) “Short” which describes a C-terminal domain truncation due to SINE Alu-Jr exonization event; 3) and “sol” which describes a soluble isoform containing only the extracellular portion.

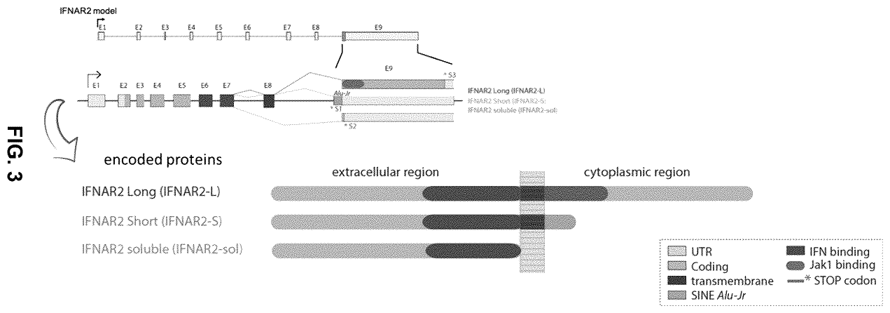

FIG. 4 shows data related to COVID-19 associated genetic variants that have been determined to be specific to the short isoform (IFNAR2-S). For sever hospitalizations, the only coding variants present in the IFNAR2-S cytoplasmic exon, with none in IFNAR2-L exons.

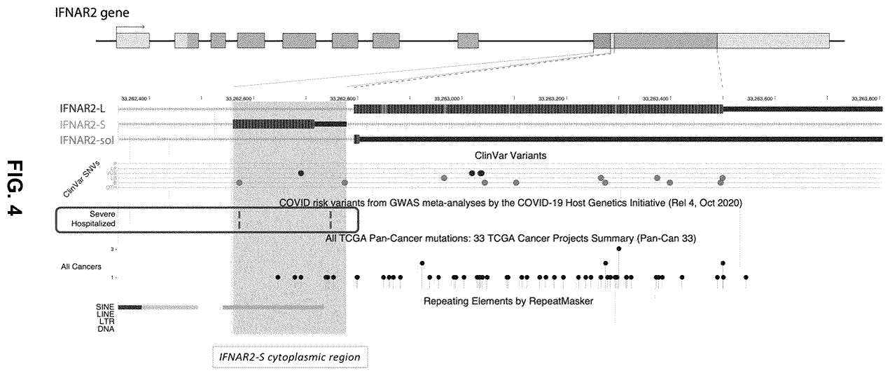

FIGS. 5A-B shows an outline of the methodology to determine the cellular function of IFNAR2-S short isoform. (A) in silico analysis of long and short RNAseq datasets; (B) in vivo analysis of IFNAR2-S and IFNAR2-L function in exemplary human cell lines, including CRISPR/Cas9 direct knock-out of one or more IFNAR2 isoforms, stable overexpression of one or more IFNAR2 isoforms, and siRNA-mediated knockdown of one or more IFNAR2 isoforms.

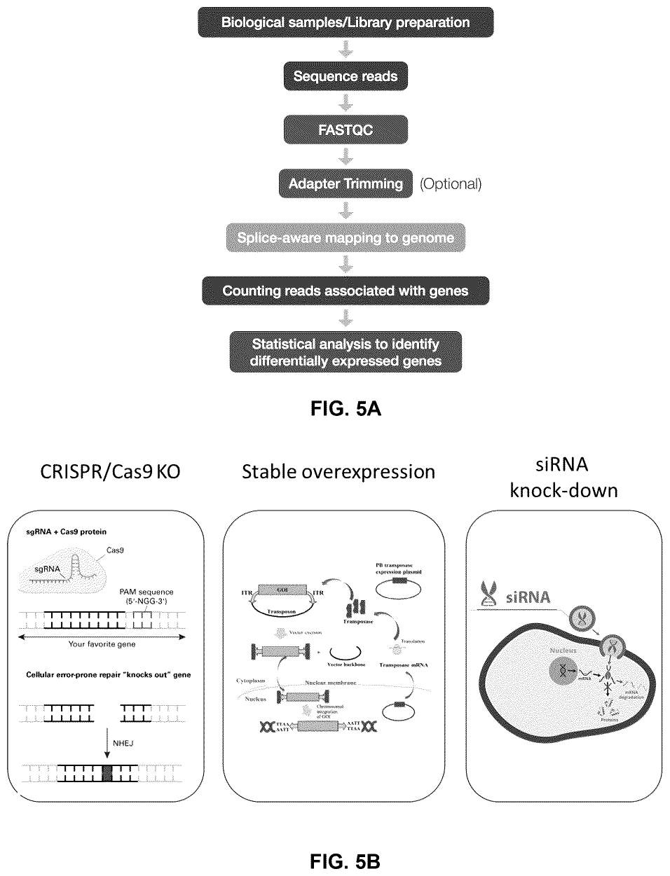

FIG. 6 shows that IFNAR2-S is the main transcribed isoform, based on isoform-specific RNA-seq analysis.

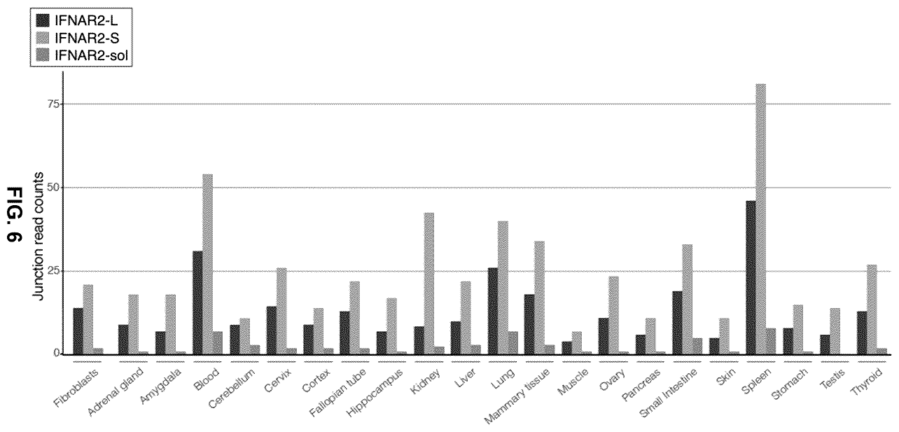

FIG. 7 shows that IFNAR2-S is the main transcribed isoform, based on isoform-specific RNA-seq analysis.

FIG. 8 show the dysregulation of IFNAR2 isoform ratio in cancer.

FIG. 9 shows dynamic changes of relative IFNAR2 isoform expression during infection.

FIG. 10 show the dynamic changes of relative IFNAR2 isoform expression during infection.

FIG. 11 show an exemplary CRISPR/Cas9 IFNAR2 editing strategy. The vignette illustrates the placement of the guides designed to generate stable HeLa mutant cell lines at the IFNAR2 locus. The double KO was generated starting from a validated IFNAR2-L clone, which was edited a second time by using the guide for the generation of IFNAR2-S KO clones. Each monoclonally expanded clone was validated by PCR and sequencing. For each KO cell line, 3 distinct clones were chosen for downstream analyses and further validated by cDNA PCR and Western Blot.

FIG. 12 show that IFNAR2-L is required for STAT phosphorylation and signaling activation.

FIG. 13 shows that IFNAR2-S knockout increases stimulation of IFN-stimulated genes.

FIG. 14 shows that IFNAR2-S knockout increases cytotoxicity in response to IFNB.

FIG. 15 shows that IFNAR2-S knockout increases cytotoxicity in response to IFNB.

FIG. 16 shows a general strategy for the stable overexpression of IFNAR2 isoforms in a PiggyBac vector to allow for stable integration into human cell lines.

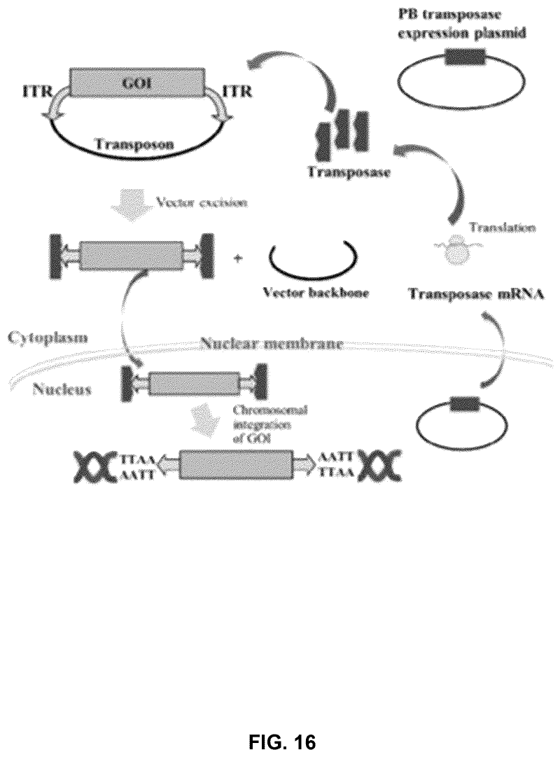

FIG. 17 show the modulation of IFNAR2-L and IFNAR2-S transcript levels in overexpression cell lines in knockout backgrounds.

FIG. 18 shows that overexpression of IFNAR2-S decreases IFN signaling.

FIG. 19 shows that overexpression of IFNAR2-S decreases IFN signaling.

FIG. 20 shows general strategy for siRNA knock-down of IFNAR2-L and -S isoforms.

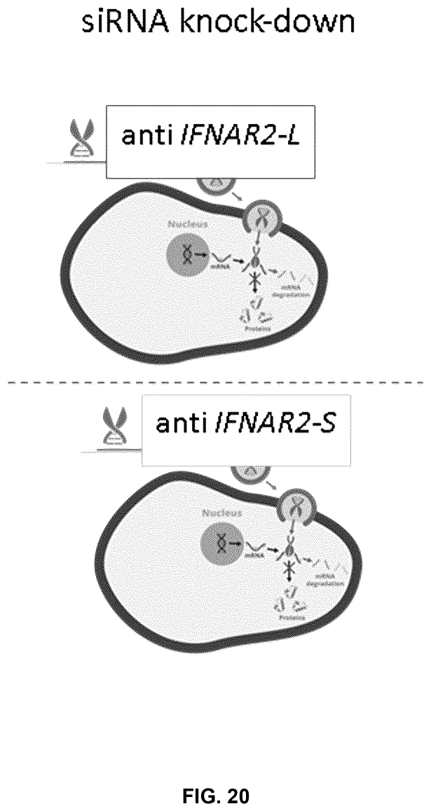

FIG. 21 shows that isoform-specific siRNA recapitulates CRISPR knockout.

FIG. 22 shows that isoform-specific siRNA recapitulates CRISPR knockout,

FIGS. 23A-B shows that IFN signaling can depend on relative ratio rather than raw abundance of IFNAR2 isoforms.

FIG. 24 CRISPR epitope tag HiBit tag was used to demonstrate that IFAR2-S is translated in HeLa Cells, and further that most IFNAR2 isoform antibodies are not isoform specific.

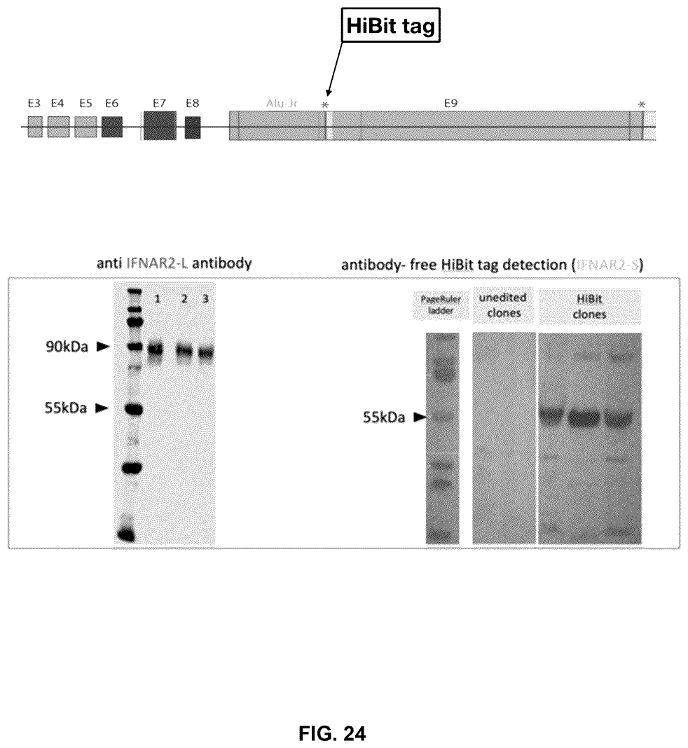

FIG. 25 shows that deletion of IFNAR2-S increase sensitivity to IFN-induced cell death.

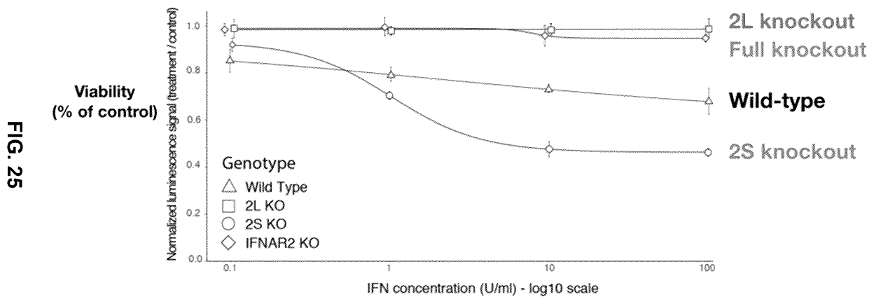

FIG. 26 shows that reintroduction of IFNAR2-S returns cells to normal IFN sensitivity.

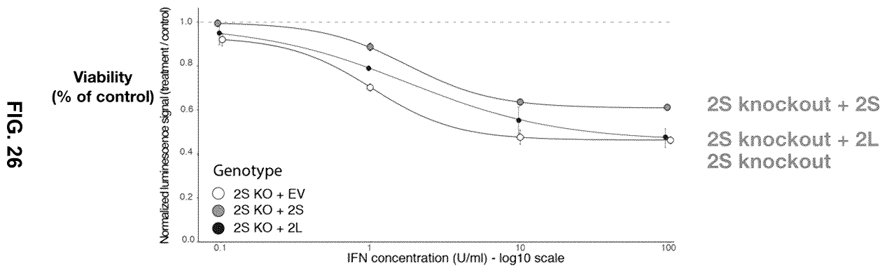

FIG. 27 shows that targeting IFNAR2S or IFNAR2L with siRNA shows expected effects and depends on siRNA dosage. (Left) cells were treated with 10 uM siRNA total, combining varying amounts of a negative scramble siRNA and an isoform-specific siRNA. (Right) Viability in response to IFN is measured by crystal violet staining. As expected, silencing the inhibitor IFNAR2S causes reduced viability.

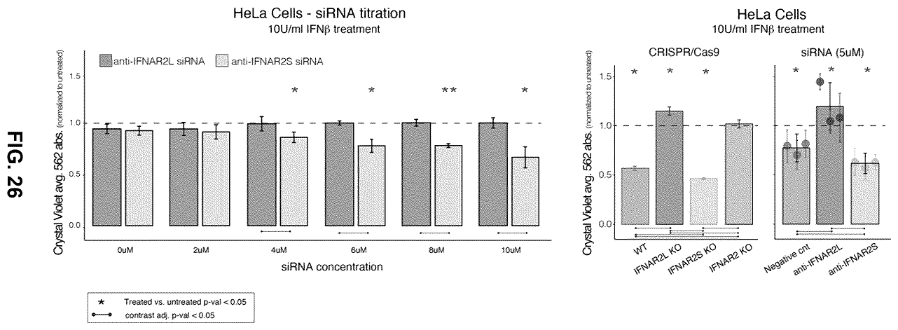

FIG. 28. schematic of working model for relative IFNAR2 isoform expression as a therapeutic application.

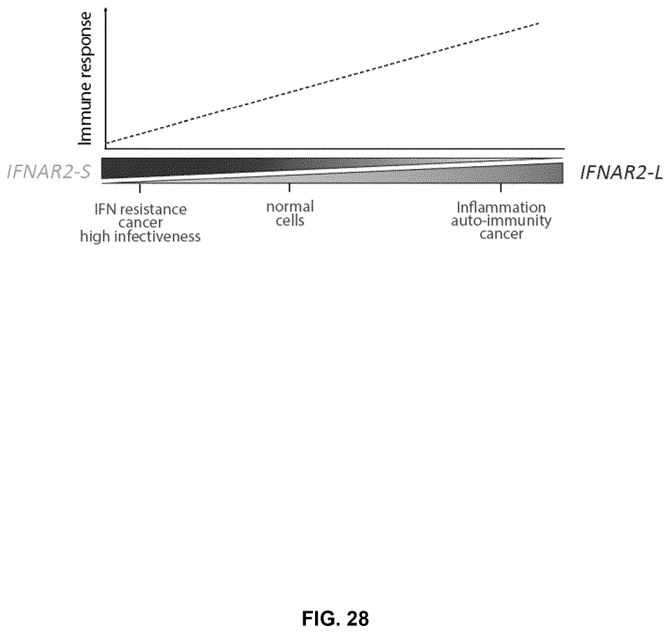

FIG. 29. IFNAR2-S ratio affects cell proliferation and cell death. Histograms show the normalized results of a crystal violet staining assessment of cell density across HeLa cell lines. Cells were transfected with a total of 10 uM siRNA, compensating target siRNA with negative control siRNA to account for potential cytotoxic effects due increasing doses of transfected siRNA. As in previous experiments, 48 hrs post transfection, cells were treated with 10 U/ml of IFNb. Four days post IFN treatment cells subjected to crystal violet staining. For each sample, we averaged absorbance values from 3 technical replicates. Significance of changes in cell density between treated and untreated (*) cells was assessed performing a pairwise t-test. Significance of changes in expression levels between treated and untreated cells across knock down cell lines at the same target siRNA molarity is reported at the bottom of the histograms and was assessed performing a pairwise emmeans contrast test in R. Based on additional replicates of the experiment, we identified 3 uM as the candidate target siRNA concentration that leads to differential cytotoxic effects of IFN.

FIG. 30. Assessment on STAT1 phosphorylation upon IFN treatment in HeLa mutant cell lines. IFNAR2 mutant HeLa cell lines were treated for 30 min with increasing doses of IFNb before harvesting. The phosphorylation of STAT1 was quantitatively assessed by phospho-Flow Cytometry.

FIG. 31A-E. (A-C) Assessment Covid19 viral replication in the absence of IFNAR2-S. We performed CRISPR/Cas9 IFNAR2 isoform specific knock out in A549 human lung carcinoma cells. Upon KO validation, cells were transfected with a plasmid for the stable expression of ACE2. (D) Cells were then pre-treated with increasing doses of IFNb (0 to 200 pM) to calculate the half maximal inhibitory concentration (IC50) of IFNb in infected cells compared to control cells. Cells were infected with 2.9×10≡PFU/well (˜11.6 moi) of the delta variant of the SARS-COV2 virus (B.1.617.2; BEI cat #NR-55672). (D) showing protective effect in Dengue model showing a decrease in viral genome replication—a proxy for infection load—upon deletion of the decoy receptor IFNAR2-S. Viral infection was assessed by RT-qPCR for the viral genome. Viral infection was performed by the Santiago lab at CU Anschutz.

### DETAILED DESCRIPTION OF THE INVENTION

In one aspect, the present inventors have characterized a novel gene product, namely an alternative IFNAR2 isoform that acts as a decoy receptor for Type I IFN, the main antiviral signaling molecule in human cells. As described below, modulating levels of this protein isoform can module cell response to IFN. In a preferred embodiment, the alternative IFNAR2 isoform includes IFNAR2-S. As noted above, characterizing IFNAR2-S as a decoy receptor has potentially transformative implications for understanding and therapeutically manipulating IFN signaling. Decades of IFN research have assumed that all detected IFNAR2 protein is produced by the long IFNAR2-L transcript. However, as a decoy receptor, the short IFNAR2-S appears identical at the surface of the cell membrane and can bind IFNB, but does not transduce IFN signaling. The present inventors have further demonstrated a previously hidden primate-specific regulatory axis, where the ratio of short/long IFNAR2 isoforms is a primary determinant of cellular IFN sensitivity. Indeed, as described herein, the manipulation of the IFNAR2 isoform ratio can be effectively used to control cellular IFN sensitivity.

As noted above, the IFNAR2 gene (SEQ ID NO. 1) encodes one of the main receptors for the Type I IFN antiviral response. The present inventors have characterized an alternative truncated isoform of IFNAR2, namely IFNAR2-S, and discovered that it acts as a negative regulator of the full-length, canonical isoform, IFNAR2-L. Modulating this isoform by overexpression or silencing is a new strategy to modulate IFN responses and may have significant therapeutic potential.

In one embodiment, the invention may include measuring, and manipulating the ratio between short and long IFNAR2 isoforms, to control cellular sensitivity to type I IFN signaling. As described herein, the expression ratio of IFNAR2 long and short isoforms as both a novel biomarker and determinant of cellular sensitivity to IFN signaling. Therefore, measuring the IFNAR2 short/long isoform ratio from cellular RNA may enable improved prediction of cellular IFN sensitivity. It will also inform downstream manipulation of the IFNAR2 isoform ratio using genetic methods (CRISPR, siRNA, oligos) for the purposes of therapeutically modulating or restoring cellular sensitivity to IFN.

In one preferred embodiment, the invention may include systems, methods, and compositions to measure IFNAR2 isoform expression ratio from cellular RNA as a biomarker of IFN sensitivity. To measure IFNAR2 isoform ratio from cellular RNA, RT-qPCR using isoform-specific primers was performed to obtain expression levels for IFNAR2-S and IFNAR2-L isoforms. The IFNAR2 short/long levels may then be used to predict cellular sensitivity to IFN, where more abundant short isoforms predicts reduced sensitivity, and more abundant long isoform predicts increased sensitivity.

In another preferred embodiment, the invention may include systems, methods, and compositions to manipulate IFNAR2 short/long isoform levels to control cellular IFN sensitivity. To manipulate the IFNAR2 isoform ratio in human cells, isoform-specific genetic targeting is used to knock out or silence the specific exon and/or splice-sites that distinguish the long and short isoforms. Multiple methods for delivering isoform-specific gene targeting therapies may be used to control IFNAR2 isoform ratio and cellular IFN sensitivity.

For example, in one embodiment, CRISPR-mediated deletion of exons can be used to decrease or ablate isoform-specific expression levels. In this embodiment, co-delivery of Cas9 and paired guide RNAs generate genomic deletions of an exon specific to either IFNAR2-L or IFNAR2-S. In another embodiment, isoform expression may be downregulated through RNA interference.

In this embodiment, siRNA sequences complementary to long- or short-specific exons may be used to decrease expression of specific isoforms to achieve a desired ratio. Examples of siRNA (sense and antisense) sequences are provided in SEQ ID NO.'s 8-1773.

In one preferred embodiment, the siRNA (sense and antisense) sequences can be directed to short-specific exons of IFNAR2, such that they decrease expression of IFNAR2-S. Exemplary, siRNA targeting IFNAR2-S can be selected from: 1438-1773.

In another preferred embodiment, the siRNA (sense and antisense) sequences directed to long- or short-specific exons may be used to decrease expression of specific isoforms, and can be selected from:

1) Anti IFNAR2-L sense:
(SEQ ID NO: 272)
GAUGAAAGUGAUAGCGAUA
1) Anti IFNAR2-L antisense:
(SEQ ID NO: 1173)
UAUCGCUAUCACUUUCAUC
2) Anti IFNAR2-L sense:
(SEQ ID NO: 214)
AGAAGAAAGUGUGGGAUUA
2) Anti IFNAR2-L antisense:
(SEQ ID NO: 1231)
UAAUCCCACACUUUCUUCU
1) Anti IFNAR2-S sense:
(SEQ ID NO: 1635)
AAACAGUCGUCCUGCCUAA
1) Anti IFNAR2-S antisense:
(SEQ ID NO: 3635)
UUAAGGGAGACUUUAUUAC
2) Anti IFNAR2-S sense:
(SEQ ID NO: 1506)
GCUGGAAUGCAGUGGCUAU
2) Anti IFNAR2-S antisense:
(SEQ ID NO: 1705)
AUAGCCACUGCAUUCCAGC

Notably, as specifically describe herein, disclosure of a sense strand also explicitly and implicitly claims its corresponding antisense strand. Conversely, any disclosure made herein of an antisense nucleotide sequence or strand, also explicitly and implicitly claims its corresponding nucleotide sequence or strand.

In another preferred embodiment, anti-sense sequences complementary to long- or short-specific exons may be used to decrease expression of specific isoforms to achieve a desired ratio. Examples of 20 base pair (bp) and 21 bp anti-sense oligonucleotides (ASOs) sequences are provided in SEQ ID NO.'s 1774-3533.

In one preferred embodiment, the ASOs sequences can be directed to short-specific exons of IFNAR2, such that they decrease expression of IFNAR2-S. Exemplary, ASOs targeting IFNAR2-S can be selected from: 3201-3533.

In another embodiment, isoform expression may be downregulated through splice-switching oligos (SSOs) targeting short or long isoform-specific splice acceptor sites used to mediate preferential splicing to occur for either IFNAR2-L or IFNAR2-S. Exemplary SSOs directed to IFNAR2-L or IFNAR2-S are provided in SEQ ID NO.'s 3149-3171, 3502, and 3504-3634.

In one preferred embodiment, the siRNA (sense and antisense) sequences can be directed to the specific terminal exon of the short IFNAR2 isoform, such that they decrease expression of IFNAR2-S. Exemplary, SSOs targeting IFNAR2-S can be selected from: 3502, and 3504-3634.

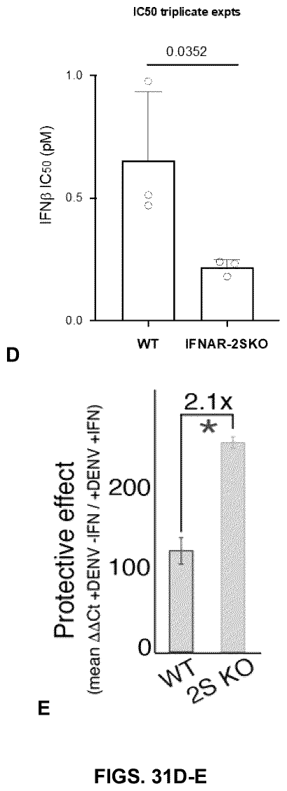

In another embodiment, the invention include novel systems, methods and compositions to increase the antiviral response in a cell through the inhibition of IFNAR2-S. Specifically, the depletion of IFNAR2-S of a target cell resulted in the decrease in viral genome replication of both SARS-CoV-2 in human A549 cells and Dengue virus in human HeLa cells, which would be understood by those of ordinary skill in the art as a proxy for viral infection load. As shown in FIG. 31, the present inventors generated a CRISPR/Cas9 IFNAR2-S isoform specific knock out in A549 human lung carcinoma cells and HeLa human cervical cancer cells. Upon validation of the knock-out, A549 cells were transfected with a plasmid for the stable expression of a SARS-CoV-2 receptor, namely the ACE2 receptor. The present inventors then pre-treated the cells with increasing doses of IFNβ (0 to 200 pM) and calculated the half maximal inhibitory concentration (IC50) of IFNβ in infected cells compared to control cells. Similar viral challenges were conducted using Dengue (DENV) virus as another exemplary model in an IFNAR2-S knock-out in HeLa cells. Again, as shown in FIG. 31 A-E, the IFNAR2-S knockout cells showed stronger antiviral response to both SARS-CoV-2 and Dengue virus infection, specifically when pre-treated with IFN prior to infection. These data show that IFNAR2-S modulation can significantly affect the antiviral response, and specifically depletion of IFNAR2-S increases the cell's antiviral response.

As a result, in one embodiment, the invention include novel methods and compositions for increasing the anti-viral response in a cell, comprising inhibiting the expression or activity of IFNAR2-S, for example through the targeted application of an RNAi reaction configured to downregulation IFNAR2-S expression in a cell. In another preferred embodiment, the invention include novel methods and compositions for treating a viral infection in a subject in need thereof. In a preferred embodiment, the invention includes inhibiting the expression or activity of IFNAR2-S in a subject in need thereof, for example through the administration of an interfering RNA molecule targeting IFNAR2-S. In a preferred embodiment, the invention includes inhibiting the expression or activity of IFNAR2-S in a subject in need thereof, for example through the administration of a pharmaceutical composition containing at least one interfering RNA molecule targeting IFNAR2-S, and a pharmaceutically acceptable carrier. In one embodiment, the RNA interfering molecule many be selected from SEQ ID NO.'s 1-3635. In a preferred embodiment, the RNA interfering molecule and can be selected from:

1) Anti IFNAR2-S sense:
(SEQ ID NO: 1635)
AAACAGUCGUCCUGCCUAA
1) Anti IFNAR2-S antisense:
(SEQ ID NO: 3635)
UUAAGGGAGACUUUAUUAC
2) Anti IFNAR2-S sense:
(SEQ ID NO: 1506)
GCUGGAAUGCAGUGGCUAU
2) Anti IFNAR2-S antisense:
(SEQ ID NO: 1705)
AUAGCCACUGCAUUCCAGC

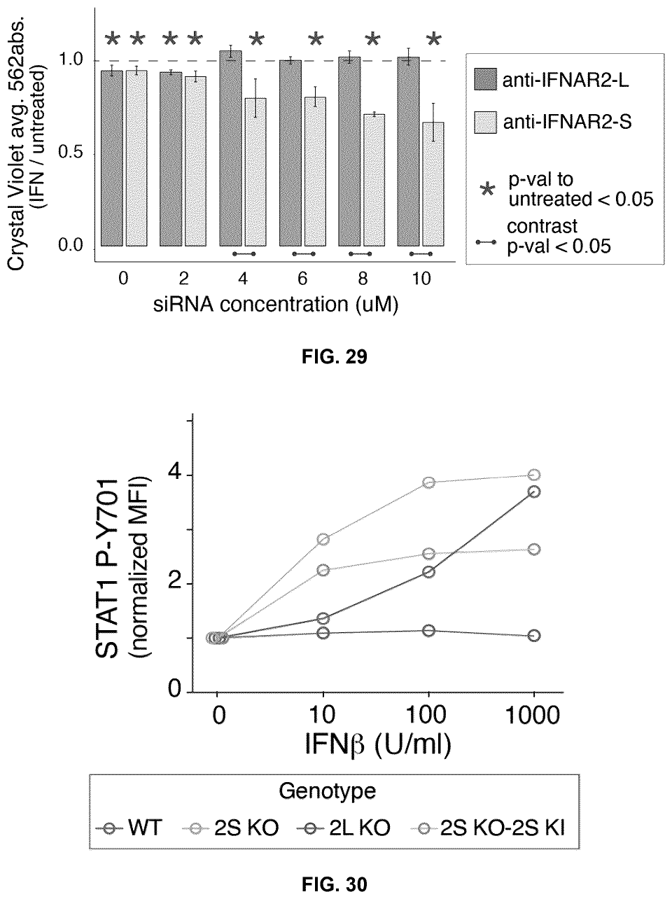

In another embodiment, the activity of INF in a call can be increased, and specifically the cytotoxic activity of INF in response to the ratio of IFNAR2-S and IFNAR2-L in a target cell. For example as show in FIG. 29, the present inventors transfected HeLa cells with increasing doses of isoform-specific siRNA up to 10 uM concentration selected from:

1) Anti IFNAR2-S sense:
(SEQ ID NO: 1635)
AAACAGUCGUCCUGCCUAA
1) Anti IFNAR2-S antisense:
(SEQ ID NO: 3635)
UUAAGGGAGACUUUAUUAC
2) Anti IFNAR2-S sense:
(SEQ ID NO: 1506)
GCUGGAAUGCAGUGGCUAU
2) Anti IFNAR2-S antisense:
(SEQ ID NO: 1705)
AUAGCCACUGCAUUCCAGC

As shown, 48 hrs post transfection, cells were treated with 10 U/ml of IFNb. Four days post IFN treatment cell viability was measured through crystal violet staining. For each sample, the inventors averaged crystal violet absorbance values from 3 technical replicates. Again, as shown in FIG. 29, the inhibition of IFNAR2-S or of IFNAR2-L had opposing effects on proliferation and cell death. The present inventors demonstrated that approximately a 3 uM concentration of the candidate target siRNA is sufficient to lead to differential cytotoxic effects of IFN. In another embodiment, a RNA interfering molecule can be contacted with a cell to inhibit IFNAR2-S, thereby increasing the relative abundance of IFNAR2-L, wherein the RNA and can be selected from: SEQ ID NO.'s 1-3635.

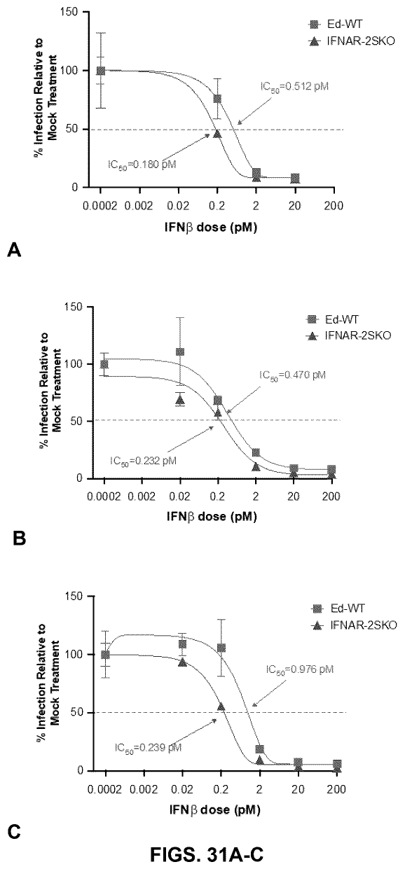

In another embodiment, the inhibition of IFNAR2-S, can cause an increase in INF signaling in a cell. As shown in FIG. 30, the present inventors generated a IFNAR2-S knock-out cell line which was treated with increasing doses of IFNb. The cells were harvested and phosphorylation of STAT1 was quantitatively assessed by phospho-Flow Cytometry. Again, as shown in FIG. 30, the IFNAR2-S KO cells reach cytokine saturation at ˜100 U/ml of IFNb. Notably, higher doses of IFNb are required to reach signal saturation in the presence of the decoy receptor IFNAR2-S in wild-type cells, while reintroduction of IFNAR2-S in IFNAR2-S knock-out cells lowers cell responsiveness to IFNb. These data confirm that IFNAR2-L is required for type I IFN signaling, and that inhibition of the IFNAR2-S decoy isoform increase the sensitivity to INF in the cell.

The terms “antisense oligomer” and “ASO” and “antisense oligonucleotide” are used interchangeably and refer to a sequence of cyclic nucleotides, each bearing a base-pairing moiety, linked by internucleotide linkages that allow the base-pairing moieties to hybridize to a target sequence in a nucleic acid (typically an RNA) by Watson-Crick base pairing, to form a nucleic acid:oligomer heteroduplex within the target sequence. The cyclic subunits are based on ribose or another pentose sugar or, in alternative embodiments, a thiomorpholino group. The oligomer may have exact or near sequence complementarity to the target sequence; variations in sequence near the termini of an oligomer are generally preferable to variations in the interior. In these methods, the antisense oligomer can be designed to block or inhibit translation of mRNA or to inhibit natural pre-mRNA splice processing and may be said to be “directed to” or “targeted against” a target sequence with which it hybridizes. It will be obvious to one skilled in the art that additional oligomer chemistries can be used to practice the invention including phosphorodiamidate-linked morpholino oligomers (PMO) or locked nucleic acid (LNA) oligomers.

Also included are vector delivery systems that are capable of expressing the oligomeric sequences of the present invention, such as vectors that express a polynucleotide sequence comprising any one or more of the sequences shown in SEQ ID NO.'s 1-3634, and variants thereof, as described herein.

The terms “vector” or “nucleic acid construct” as used herein means a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof or be integrated with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

A “pharmaceutical composition” or “pharmaceutical composition of the invention” refers to a compound of the invention or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional antibiotic, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

The term “pharmaceutically acceptable carrier” as used herein further pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, diluents, excipients, etc. can be found in standard pharmaceutical texts. See, for example, “Handbook of Pharmaceutical Additives,” 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

Suitable pharmaceutically acceptable carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition of the invention may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages. Exemplary parenteral administration forms include solutions or suspensions of active compounds in sterile aqueous solutions, for example, aqueous propylene glycol or dextrose solutions. Such dosage forms may be suitably buffered, if desired.

A pharmaceutical composition of the invention may be administered as single or multiple agents, for example a pharmaceutical composition of a the compound of the invention, or a pharmaceutical composition of the compound of the invention and a second therapeutic compound or agent. In some embodiments, the methods the pharmaceutical composition of the invention can be used to treat a mitochondrial disease, or one or more of its symptoms. Pharmaceutical compositions suitable for the delivery of the compound of the invention as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

“Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition associated with the IFN signaling pathway, such as cancer, or a viral infection, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

Treatment with an antisense oligonucleotides of the invention may modulate the relative expression levels of cellular IFNAR2-L or IFNAR2-S in a subject to be treated. In a preferred embodiment, this expression of IFNAR2-S in inhibited, while in alternative embodiments expression of IFNAR2-L is inhibited. In alternative embodiment, expression of IFNAR2-S in upregulated, for example by overexpression, while in alternative embodiments, expression of IFNAR2-L in upregulated, for example by overexpression. In other embodiments, expression of IFNAR2-S in upregulated, while expression of IFNAR2-L in downregulated, and vice versa.

Treatment with an antisense oligonucleotides of the invention may modulate the relative ratio of cellular IFNAR2-L or IFNAR2-S in a subject to be treated. In a preferred embodiment, this ratio may be modulated such that there more IFNAR2-L than IFNAR2-S present in the cell. In another preferred embodiment, this ratio may be modulated such that there is less IFNAR2-L than IFNAR2-S present in the cell. In further preferred embodiments, this ratio may be modulated such that there is approximately the same amount of IFNAR2-L as IFNAR2-S present in the cell.

Treatment may be effectuated by a therapeutically effective amount of one or more oligonucleotide of the invention An “effective amount” or “therapeutically effective amount” refers to an amount of therapeutic compound, such as an antisense oligonucleotide, administered to a human subject, either as a single dose or as part of a series of doses, which is effective to produce a desired therapeutic effect. For an antisense oligonucleotide, this effect is typically brought about by inhibiting translation or natural splice-processing of a selected target sequence. An effective amount may be variable such as 5 mg/kg of a composition comprising a thiomorpholino antisense oligonucleotide for a period of time to treat the subject. In one embodiment, an effective amount might be 5 mg/kg of a composition comprising an oligonucleotide, such as an siRNA, ASO or SSO, to modulate the relative ratio of IFNAR2-L and IFNAR2-S present in the cell.

When the oligonucleotides of this invention are administered as pharmaceutical compositions, to humans and animals, they can be given per se or as a pharmaceutical composition containing, for example, 0.1 to 99% (more preferably, 10 to 30%) of active ingredient in combination with a pharmaceutically acceptable carrier. As noted above, the formulations or preparations of this disclosure may be given orally, parenterally, systemically, topically, or intramuscular administration. They are typically given in forms suitable for each administration route. For example, they are administered in tablets or capsule form, by injection, inhalation, eye lotion, ointment, suppository, etc. administration by injection, infusion or inhalation; topical by lotion or ointment.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the oligomers of the present invention, which may be used in a suitable hydrated form, and/or the pharmaceutical compositions of the present invention, may be formulated into pharmaceutically-acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular oligomer of this disclosure employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and gradually increase the dosage until the desired effect is achieved. In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular, intramuscular and subcutaneous doses of the compounds of this invention for a patient, when used for the indicated effects, will range from about 0.0001 to about 100 mg per kilogram of body weight per day.

Preferred doses of the oligonucleotides of the invention are administered generally from about 5-100 mg/kg. In some cases, doses of greater than 100 mg/kg may be necessary. For i.v. administration, preferred doses are from about 0.1 mg to 100 mg/kg. In some embodiments, the thiomorpholino oligomers are administered at doses of about 2 mg/kg, to about 100 mg/kg, including all integers in between.

If desired, the effective daily dose of the active compound may be administered as two, three, four, five, six or more sub-doses administered separately at appropriate intervals throughout the day, optionally, in unit dosage forms. In certain situations, dosing is one administration per day. The dosing frequency is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to maintain the desired expression levels of IFNAR2-L and IFNAR2-S in the subject.

In some embodiments, the oligonucleotides of the invention are administered, generally at regular intervals (e.g., daily, weekly, biweekly, monthly, bimonthly). The oligomers may be administered at regular intervals, e.g., daily; once every two days; once every three days; once every 3 to 7 days; once every 3 to 10 days; once every 7 to 10 days; once every week; once every two weeks; once monthly. For example, the oligomers may be administered once weekly by intravenous infusion. The oligomers may be administered intermittently over a longer period, e.g., for several weeks, months or years. For example, the oligomers may be administered once every: one, two, three, four, five, six, seven, eight, nine, ten, eleven or twelve months. In addition, the oligomers may be administered once every: one, two, three, four or five years. Administration may be followed by, or concurrent with, administration of an antibiotic, steroid or other therapeutic agent. The treatment regimen may be adjusted (dose, frequency, route, etc.) as indicated, based on the results of immunoassays, other biochemical tests and physiological examination of the subject under treatment.

Nucleic acid molecules can be administered to cells by a variety of methods known to those familiar to the art, including, but not restricted to, encapsulation in liposomes or lipoplexes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Among other benefits, microemulsification provides enhanced bioavailability by preferentially directing absorption to the lymphatic system instead of the circulatory system, which thereby bypasses the liver, and prevents destruction of the compounds in the hepatobiliary circulation.

While all suitable amphiphilic carriers are contemplated, the presently preferred carriers are generally those that have Generally-Recognized-as-Safe (GRAS) status, and that can both solubilize the compound of this disclosure and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethylene-glycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or monounsaturated fatty acids (SPAN-series) or corresponding ethoxylated analogs (TWEEN-series).

The delivery may occur by use of liposomes, lipoplexes, nanocapsules, microparticles, microspheres, lipid particles, vesicles, and the like, for the introduction of the compositions of this disclosure into suitable host cells. In particular, the compositions of this disclosure may be formulated for delivery either encapsulated in a lipid particle, a liposome, a lipoplex, a vesicle, a nanosphere, a nanoparticle, or the like. The formulation and use of such delivery vehicles can be carried out using known and conventional techniques.

Hydrophilic polymers which may be suitable for use in this disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

A formulation of this disclosure may comprise a biocompatible polymer selected from the group consisting of polyamides, polycarbonates, polyalkylenes, polymers of acrylic and methacrylic esters, polyvinyl polymers, polyglycolides, polysiloxanes, polyurethanes and copolymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letters alpha, beta, or gamma, respectively. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds. The complexation takes place by Van der Waals interactions and by hydrogen bond formation. The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 micrometers in diameter; large unilamellar vesicles (LUVS) are typically larger than 0.05 micrometers Oligolamellar large vesicles and multilamellar vesicles have multiple, usually concentric, membrane layers and are typically larger than 0.1 micrometers. Liposomes with several nonconcentric membranes, i.e., several smaller vesicles contained within a larger vesicle, are termed multivesicular vesicles. Thus, formulations comprising liposomes containing a thiomorpholino oligomer of this disclosure, where the liposome membrane is formulated to provide a liposome with increased carrying capacity. Alternatively, or additionally, the compound of this disclosure may be contained within, or adsorbed onto, the liposome bilayer of the liposome. An oligomer of this disclosure may be aggregated with a lipid surfactant and carried within the liposome's internal space; in these cases, the liposome membrane is formulated to resist the disruptive effects of the active agent-surfactant aggregate. The lipid bilayer of these liposomes may contain lipids derivatized with a saccharide, including a disaccharide such as lactose, a polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of this disclosure are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes of the present invention.

Liposomes according to this disclosure may be prepared by any of a variety of techniques that are known in the art. See, e.g., U.S. Pat. No. 4,235,871; Published PCT applications WO 96/14057; New RRC, Liposomes: A practical approach, TRL Press, Oxford (1990), pages 33-104; Lasic D D, Liposomes from physics to applications, Elsevier Science Publishers BV, Amsterdam, 1993. For example, liposomes of this disclosure may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT™ and Lipofectamine™ may be utilized to introduce polynucleotides or proteins into cells.

The release characteristics of a formulation of this disclosure depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween™ and Pluronic™. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

In addition to the methods provided herein, the oligomers for use according to the invention may be formulated for administration in any convenient way for use in human or veterinary medicine, by analogy with other pharmaceuticals. The oligomers of the invention and their corresponding formulations may be administered alone or in combination with other therapeutic strategies in the treatment of a disease or condition that would be responsive to reduced or increased sensitivity to IFN, mediated by the relative cellular expression levels of IFNAR2-L or IFNAR2-S in a subject in need thereof.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual Cold Spring Harbor Laboratory Press, Cold Spring Harbor, N.Y. and Current Protocols in Molecular Biology (Ausbel et al., eds., John Wiley & Sons, Inc., As appropriate, procedures involving the use of commercially available kits and reagents are generally carried out in accordance with manufacturer defined protocols and/or parameters unless otherwise noted.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “expression,” as used herein, or “expression of a coding sequence” (for example, a gene or a transgene) refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. It should be noted that any reference to a SEQ ID, or sequence specifically encompasses that sequence, as well as all corresponding sequences that correspond to that first sequence. For example, for any amino acid sequence identified, the specific specifically includes all compatible nucleotide (DNA and RNA) sequences that give rise to that amino acid sequence or protein, and vice versa.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

A polynucleotide sequence is operably linked to an expression control sequence(s) (e.g., a promoter and, optionally, an enhancer) when the expression control sequence controls and regulates the transcription and/or translation of that polynucleotide sequence. Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides.

As described herein, an “immune response” may typically either be a specific reaction of the adaptive immune system to a particular antigen (so called specific or adaptive immune response) or an unspecific reaction of the innate immune system (so called unspecific or innate immune response). The invention relates to the core to specific reactions (adaptive immune responses) of the adaptive immune system. Particularly, it relates to adaptive immune responses to infections by viruses like e.g. COVID-19 coronaviruses. However, this specific response can be supported by an additional unspecific reaction (innate immune response). Therefore, the invention also relates to a compound for simultaneous stimulation of the innate and the adaptive immune system to evoke an efficient adaptive immune response.

As used herein, the term “innate immune system,” or “innate immunity” also known as non-specific immune system, comprises the cells and mechanisms that defend the host from infection by other organisms in a non-specific manner. This means that the cells of the innate system recognize and respond to pathogens in a generic way, but unlike the adaptive immune system, it does not confer long-lasting or protective immunity to the host. The innate immune system may be e.g. activated by ligands of pathogen-associated molecular patterns (PAMP) receptors, e.g. Tol like receptors (TLRs) or other auxiliary substances such as lipopolysaccharides, TNF-alpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR10, TLR11, TLR12 or TLR13, a ligand of a NOD-like receptor, a ligand of a RIG-I like receptor, an immunostimulatory nucleic acid, an immunostimulatory RNA (isRNA), a CpG-DNA, an antibacterial agent, or an anti-viral agent. Typically a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system through a process known as antigen presentation; and/or acting as a physical and chemical barrier to infectious agents.

An “exon” refers to a defined section of nucleic acid that encodes a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human IFNAR2 gene has 9 exons. An “intron” refers to a nucleic acid region (within a gene) that is not translated into a protein. An intron is a non-coding section that is transcribed into a precursor mRNA (pre-mRNA), and subsequently removed by splicing during formation of the mature RNA.

In certain embodiments, the relative expression and/or ratio of isoforms IFNAR2-L or IFNAR2-S may be a biomarker to predict cellular sensitivity to IFN. For example, as noted above abundant short isoform predicts reduced sensitivity to IFN, while more abundant long isoform predicts increased sensitivity to IFN. Such As used herein, a biological marker (“biomarker” or “marker”) is a characteristic that is objectively measured and evaluated as an indicator of normal biologic processes, pathogenic processes, or pharmacological responses to therapeutic interventions, consistent with NIH Biomarker Definitions Working Group (1998). Markers can also include patterns or ensembles of characteristics indicative of particular biological processes. The biomarker measurement can increase or decrease to indicate a particular biological event or process. In addition, if the biomarker measurement typically changes in the absence of a particular biological process, a constant measurement can indicate occurrence of that process.

As used herein, the phrase “gene expression” or “protein expression,” such as the level of “IFNAR2-L or IFNAR2-S gene expression,” or “the level of IFNAR2-L or IFNAR2-S protein expression,” includes any information pertaining to the amount of gene transcript or protein present in a sample, in a cell, in a patient, secreted in a sample, and secreted from a cell as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

As used herein, a the term “RNA interference molecule” or “RNAi molecule” means a RNA molecules that can modulate the expression of a target gene, for example through an RNAi-mediated reaction in a cell, for example by an siRNA, or through an alternative splicing event in a cell for example by an SSO, or through steric hinderance, for example by an ASO.

As used herein, “inhibit, “inhibition,” “suppress,” “downregulate” or “silencing” refers to partial or complete loss-of-function through targeted inhibition of gene expression in a cell and may also be referred to as “knock down,” of IFNAR2-L or IFNAR2-S preferably through an RNAi pathway response or through an endonuclease-mediated knockout, such as a CRISPR/Cas9-mediated knock-out of IFNAR2-L or IFNAR2-S. Depending on the circumstances and the biological problem to be addressed, it may be preferable to partially reduce gene expression or IFNAR2-L or IFNAR2-S, or both. Alternatively, it might be desirable to reduce gene expression as much as possible. The extent of silencing may be determined by any method known in the art, some of which are summarized in International Publication No. WO 99/32619, incorporated herein by reference. As used herein, “inhibit, “inhibition,” “suppress,” “downregulate” or “silencing” of the level or activity of an agent, such as, for example, a preRNA, mRNA, rRNA, tRNA, snoRNA, snRNA expressed by the target gene, and/or of the protein product encoded by it, means that the amount is reduced by 10% or more, for example, 20% or more, preferably 30% or more, more preferably 50% or more, even more preferably 70% or more, most preferably 80% or more, for example, 90%, relative to a cell or organism lacking a dsRNA molecule of the disclosure.

Moreover, the terms “enhance”, “overexpressed” generally refer to a statistically significant increase, for example in a trait, phenotype or level of actual or relative gene expression, preferably expression of IFNAR2-L or IFNAR2-S. For the avoidance of doubt, these terms generally refer to about a 5% increase in a given parameter or value, about a 10% increase, about a 15% increase, about a 20% increase, about a 25% increase, about a 30% increase, about a 35% increase, about a 40% increase, about a 45% increase, about a 50% increase, about a 55% increase, about a 60% increase, about a 65% increase, about 70% increase, about a 75% increase, about an 80% increase, about an 85% increase, about a 90% increase, about a 95% increase, about a 100% increase, or more over the control value. These terms also encompass ranges consisting of any lower indicated value to any higher indicated value, for example “from about 5% to about 50%”, etc.

The present invention may include novel systems and methods for use of a gRNA which may be utilized by the CRISPR/Cas9 system to disrupt IFNAR2-L or IFNAR2-S. Generally, CRISPR/Cas9 may be used to generate a knock-out or disrupt target genes by co-expressing a gRNA specific to the gene to be targeted and the endonuclease Cas9. CRISPR may consist of two components: gRNA and a non-specific CRISPR-associated endonuclease (Cas9). The gRNA may be a short synthetic RNA composed of a scaffold sequence that may allow for Cas9-binding and a ˜20 nucleotide spacer or targeting sequence which defines the genomic target to be modified.

As used herein, the term “antisense RNA” or “asRNA” refers to an RNAi agent that is a single stranded oligonucleotide. In a typical asRNA, the single strand is complementary to all or a part of the target mRNA. The complementarity of an asRNA may be with any part of the specific gene transcript, i.e., at the 5′ non-coding sequence, 3′ non-translated sequence, introns, or the coding sequence. asRNA may be introduced into a cell to inhibit translation of a complementary mRNA by base pairing to it and physically obstructing the translation machinery. Antisense RNA anneal to a complementary mRNA target sequence, and translation of the mRNA target sequence is disrupted as a result of steric hindrance of either ribosome access or ribosomal read through. The antisense RNA mechanism is different from RNA interference (RNAi), a related process in which double-stranded RNA fragments (dsRNA, also called small interfering RNAs (siRNAs)) trigger catalytically mediated gene silencing, most typically by targeting the RNA-induced silencing complex (RISC) to bind to and degrade the mRNA. Annealing of a strand of the asRNA molecule to mRNA or DNA can result in fast degradation of duplex RNA, hybrid RNA/DNA duplex, or duplex RNA resembling precursor tRNA by ribonucleases in the cell, or by cleavage of the target RNA by the antisense compound itself.

The term “subject” refers to any animal. In certain embodiments, the subject is a mammal. In certain embodiments, the subject is a human (e.g., a man, a woman, or a child). The human may be of either sex, or may be at any stage of development. In certain embodiments, the subject has been diagnosed with the mitochondrial condition or disease to be treated. In certain embodiments, the subject is an experimental animal (e.g., mouse, rat, rabbit, dog, pig, or primate). The experimental animal may be genetically engineered. In certain embodiments, the subject is a domesticated animal (e.g., dog, cat, bird, horse, cow, goat, sheep, or chicken).

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein and in the appended claims, the singular forms “a”, “an”, and “the” include plural referents unless the context clearly dictates otherwise. Thus, for example, reference to “a plant” includes a plurality of such plants; reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1% compared to the specifically recited value.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Experimental Rationale for the Present Invention

Although the short IFNAR2-S isoform has been assumed to be silenced and nonfunctional, re-analysis of both short- and long-read RNA-seq from human tissues revealed widespread expression of IFNAR2. Our analysis also revealed that the ratio of short and long isoforms is dynamically regulated in disease states including cancer and cells infected with viruses including SARS-CoV2. While the canonical IFNAR2-L isoform has been studied for decades, the present inventors conducted the first isoform-specific IFNAR2 knockout and silencing experiments in human cell lines. Using CRISPR on HeLa cells, the present inventors generated a series of clones with homozygous knock out deletions of exons specific to the short or long isoform, or an exon common to both isoforms (full knockout). Wild-type, IFNAR2-L knockout, IFNAR2-S knockout, and full knockouts were evaluated using a series of complementary assays for IFN signaling. These included immunoblotting for phosphorylation of STAT1/2, qPCR of IFN-stimulated gene induction, and assessment of cytotoxicity induced by IFN treatment.

As expected based on the known function of the full-length IFNAR2-L isoform, specific knockout of the IFNAR2-L exon or a full knockout of both isoforms resulted in ablation of type I IFN signaling. However, specific knockout of the short IFNAR2-S isoform resulted in an increase in type I IFN signaling. These results showed consistent trends across different methods to assay IFN signaling. These results were reproduced using isoform-specific siRNA experiments instead of CRISPR, in both HeLa and A549 cell lines. Furthermore, IFNAR2-L or IFNAR2-S were overexpressed in wild-type and knockout backgrounds, and assayed the cytotoxic effect of IFNB treatment in comparison to control cells. Overexpression of IFNAR2-L resulted in increased sensitivity (based on cell viability) to IFN signaling, while overexpression of IFNAR2-S resulted in decreased sensitivity to IFN signaling. Together, the present invention reveal a novel role for IFNAR2-S functioning as a decoy in human cells.

### Example 2: IFNAR2-S is the Main Transcribed Isoform

Junction read counts show higher splicing of the short isoform of the type I interferon receptor gene (IFNAR2-S). Histogram shows the relative abundance of the main IFNAR2 isoforms across multiple human healthy tissues and cell lines. In contrast to the prevalent view oh higher splicing of the full-length functional isoform (IFNAR2-L), exon-exon junction read counts from Illumina RNAseq datasets support higher prevalence of the alternative isoform IFNAR2-S.

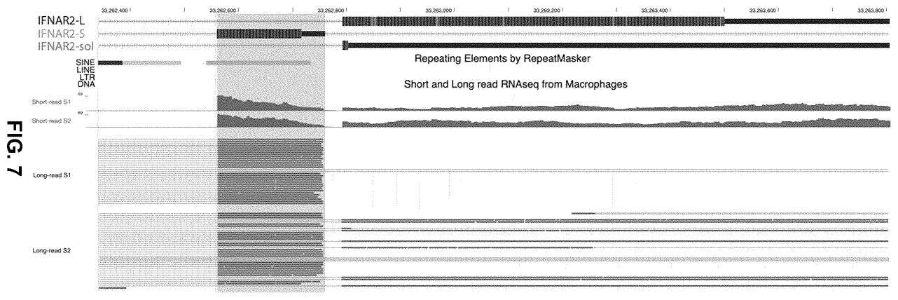

As shown in FIG. 7, Long read RNA sequencing support higher splicing of the short isoform of the type I interferon receptor gene (IFNAR2-S). The UCSC genome browser (genome.ucsc.edu) was used to analyze read coverage from matched Illumina short read RNAseq and Oxford Nanopore long read RNAseq samples over the IFNAR2-S (highlighted in orange) and IFNAR2-L genomic locus. Both sequencing strategies show that the short IFNAR2-S isoform is highly transcribed in virtually all tissues. On top, read coverage for Illumina short read RNAseq samples is shown. At the bottom, long read RNAseq for the same samples is shown as individually sequenced reads.

### Example 3: Dysregulation of IFNAR2 Isoform Ratio in Cancer

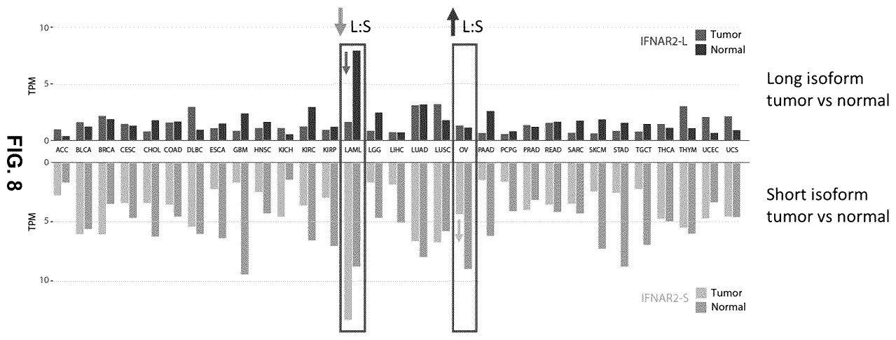

As shown in FIG. 8, alteration of the physiological IFNAR2-L to IFNAR2-S relative isoform abundance is a phenomenon in cancer. Histogram shows the relative abundance of the main IFNAR2 isoforms across multiple matched human healthy and cancer tissues and cell lines. On top, average normalized expression levels for IFNAR2-L (in blue) are reported for matched tumor (on the left in the pair, in light blue) and healthy samples (on the right In the pair, in dark blue). At the bottom, average normalized expression levels for IFNAR2-s (in orange) are reported for matched tumor (on the left in the pair, in light orange) and healthy samples (on the right In the pair, in dark orange). Isoform dysregulation was detected in both directions, either as a reduction of the L:S ratio (like in leukemic cells, LAML) or an in increased L:S ration (like in ovarian carcinomas, OV). Normalized count data (TPM, transcripts per million) were obtained from the GEPIA2 portal (http://gepia2.cancer-pku.cn/).

### Example 4: Dynamic Changes of Relative IFNAR2 Isoform Expression During Infection

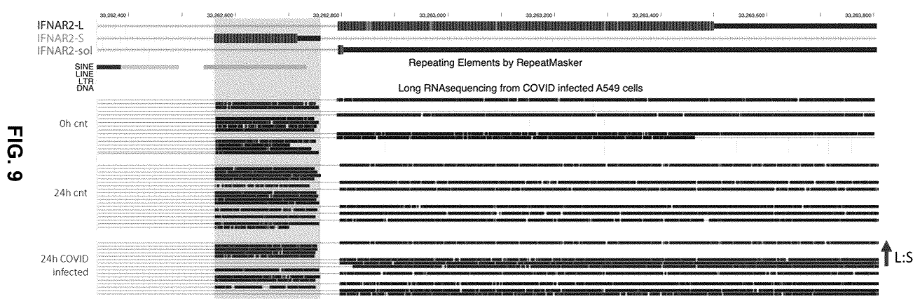

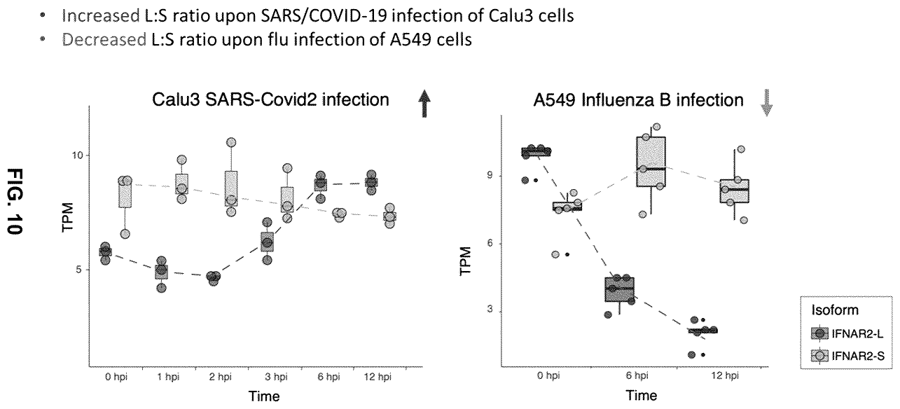

As shown in FIG. 9, long read RNA sequencing show dynamic changes in the relative IFNAR2-L to IFNAR2-S ratio upon viral infection. The UCSC genome browser (genome.ucsc.edu) was used to visualize read coverage from Oxford Nanopore long read RNA sequencing over the IFNAR2-S (highlighted in orange) and IFNAR2-L genomic locus. Control A549 cells show lower IFNAR2-L expression levels than IFNAR2-S (˜1:3 ratio). Upon infection with Covid-19, an increase in IFNAR2-L expression levels can be detected (1:1 ratio), consistent with data from Calu3 cells (FIG. 10).

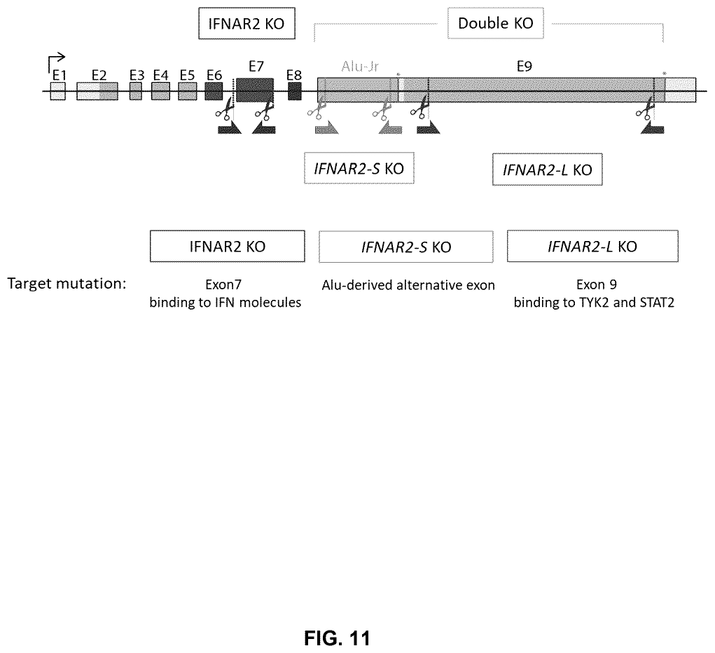

As shown in FIG. 10, Short read RNA sequencing show dynamic changes in the relative IFNAR2-L to IFNAR2-S ratio upon viral infection. Boxplots show changes in expression levels of IFNAR2-S and IFNAR2-L upon viral infection in vitro. Calu3 cells (left) have higher IFNAR2-S baseline expression levels. SARS-Covid19 infection time course analysis reveals a shift in the relative expression levels of IFNAR2-S, that slightly decreases over time, and IFNAR2-L, which increases over time. Increased levels of IFNAR2-L is consistent with observations in A549 cells infected with Covid-19 (previous slide). A contrasting trend is observed in A549 cells when infected with the Influenza B virus. FIG. 11 shows that IFNAR2-L is required for STAT phosphorylation and signaling activation and specifically the absence of phosphorylation of STAT1 and STAT2 in mutant cell lines lacking IFNAR2-L (2L-KO and double KO) or the region binding IFN (IFNAR2 KO).

### Example 5: IFNAR2-L is Required for STAT Phosphorylation and Signaling Activation

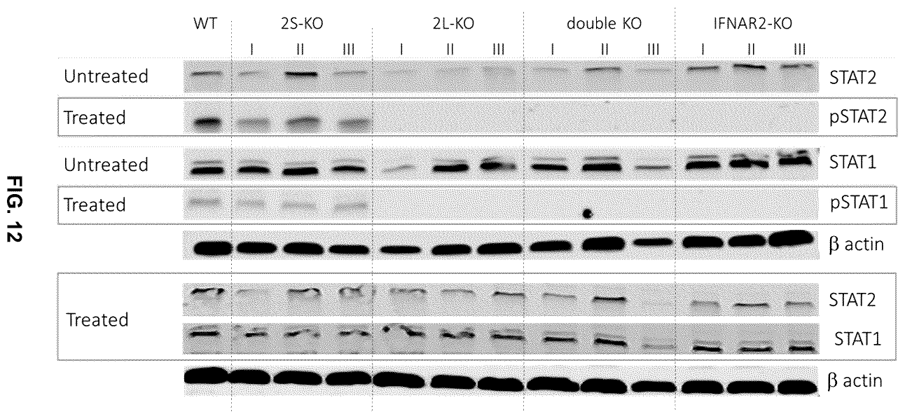

As shown in FIG. 12, the absence of phosphorylation of STAT1 and STAT2 in mutant cell lines lacking IFNAR2-L (2L-KO and double KO) or the region binding IFN (IFNAR2 KO). Assessment on STAT1 and STAT2 phosphorylation upon IFN treatment in HeLa mutant cell lines. IFNAR2 mutant HeLa cell lines were treated for 30 min with 10 U/ml of IFNb (Treated) before harvesting. ˜12 ug of whole protein lysate was loaded under denaturating conditions. Phosphorylation of STAT1 (which is bound to IFNAR1) and STAT2 (bound to IFNAR2) represent the first step in the type I IFN pathway activation. Our western blot indirectly validates our CRISPR/Cas9 editing and confirms previous observations that only in the presence of a functional IFNAR2 receptor (wild-type cells, WT, and IFNAR2-S KOs, 2S-KO) the signaling cascade is activated.

### Example 6: IFNAR2-S Knockout Increases Stimulation of IFN-Stimulated Genes

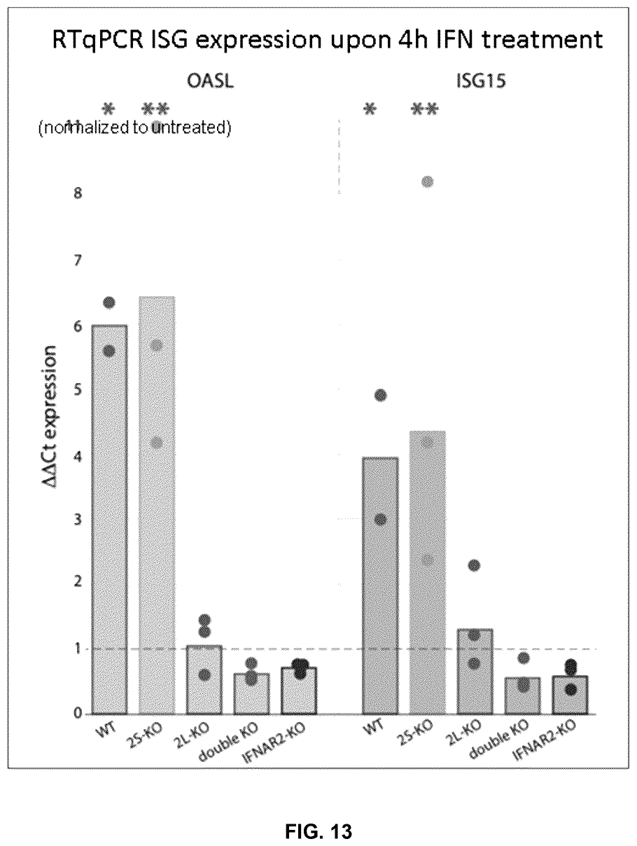

As shown in FIG. 13, knockout of canonical IFNAR2-L impairs ability to activate IFN-stimulated genes, and that knockout of IFNAR2-S leads to higher inducibility of the IFN-stimulated genes (eg, OASL and ISG15), consistent with decoy activity. Knock out of IFNAR2-S brings to higher activation of the type I IFN signaling pathway. Histogram shows the normalized expression levels of two canonical interferon stimulated genes (ISGs), OASL and ISG15 across mutant HeLa cell lines generated for this project as measured by RT-qPCR. For each cell line, 3 different clones generated by CRISPR/Cas9 genome editing were used, whereas for the wild-type (WT) we used two pseudo-biological replicates. At 4 hours post treatment with 10 U/ml of IFNb, treated and control cells expression of ISGs upon IFN stimulation in mutant cell lines that lack the decoy receptor IFNAR2-S (2S-KO, in yellow were harvested in lysis buffer. For each sample, we averaged Ct values from 3 technical replicates (dots). Normalization was first performed in respect to the Ct of the CTCF gene, here used as housekeeping (DCt), and then in respect to the matched untreated group (DDCt). Significance of changes in expression levels between treated and untreated (*) was assessed performing a chi-square test. In line with our expectation, we observed higher w) compared to wild type cells (WT, in dark grey). We saw no significant changes in ISG expression levels when the functional IFNAR2-L is deleted (2L-KO, in blue), in the double 2L-2S KO line (double KO, in purple), and in the IFN unresponsive line (IFNAR2-KO, in maroon).

### Example 7: IFNAR2-S Knockout Increases Cytotoxicity in Response to IFNB

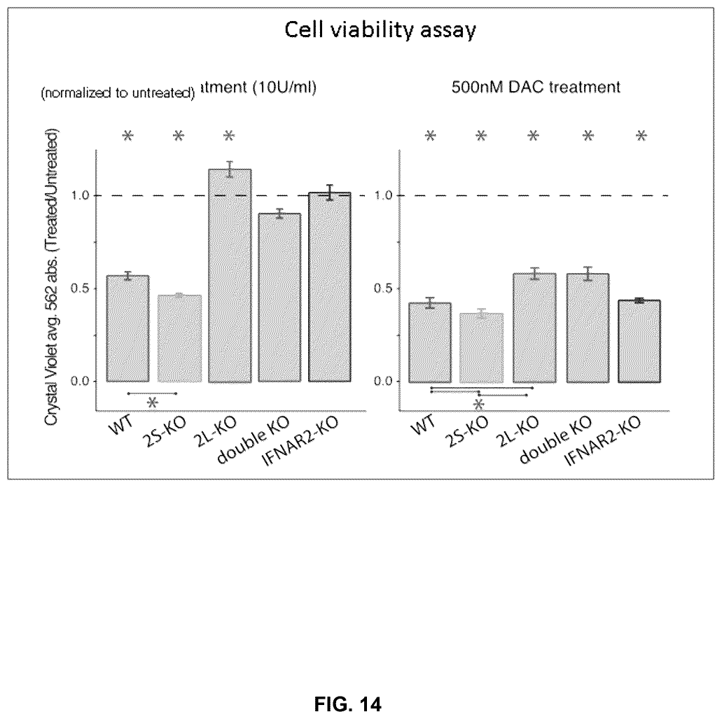

As shown in FIG. 14, knockout of IFNAR2-L impairs response to IFNB treatment and Decitabine (cytotoxic DNMT inhibitor that acts through IFN signaling), and knock out of IFNAR2-S leads to increased cell death upon IFNB treatment, compared to WT cells. Knock out of IFNAR2-S brings to less proliferation and higher cell death. Histograms show the normalized results of a crystal violet staining assessment of cell density across HeLa mutant cell lines upon treatment. Clones were grown in presence of IFNb (10 U/ml) for four days (left) or in presence of Decitabine (DAC) for six days (right). Control cells were grown with matched concentrations of PBS for the IFN treatment experiment and DMSO for the DAC treatment experiment (to match the drug resuspension media). Media was replaced after 24 h for the DAC experiment with complete culturing media to allow the cells to grow in absence of DMSO. Once control cells reached confluency, cells were passaged at a lower density, and subjected to crystal violet staining. Based on cell density in the most confluent control wells, the dilution factor was optimized and then kept constant for all clones in the same experiment (i.e., if 1:100 dilution factor (10 ul of cell suspension) was established based on the representative well, all wells for the same experiment were seeded at a 1:100 dilution, regardless of the individual cell density). For each sample, we averaged absorbance values from 3 technical replicates. Significance of changes in cell density between treated and untreated (*) cells was assessed performing a pairwise t-test. Significance of changes in expression levels between treated and untreated cells across cell lines is reported at the bottom of the histograms and was assessed performing a pairwise emmeans contrast test in R. In line with our expectation, we observed reduced cell density in the wild-type and the IFNAR2-S KO cell line (2S-KO, in yellow), and a further significant reduction when the 2S-KO is compared to wild type cells (WT, in dark grey). We saw no significant changes in cell density when the functional IFNAR2-L is deleted (2L-KO, in blue), in the double 2L-2S KO line (double KO, in purple), and in the IFN unresponsive line (IFNAR2-KO, in maroon) in the IFN treatment experiment. Given the broader target spectrum of the DAC drug, we detected reduced cell density in all cell lines; however, trends were still consistent with the IFN treated cells.

### Example 8: IFNAR2-S Knockout Increases Cytotoxicity in Response to IFNB

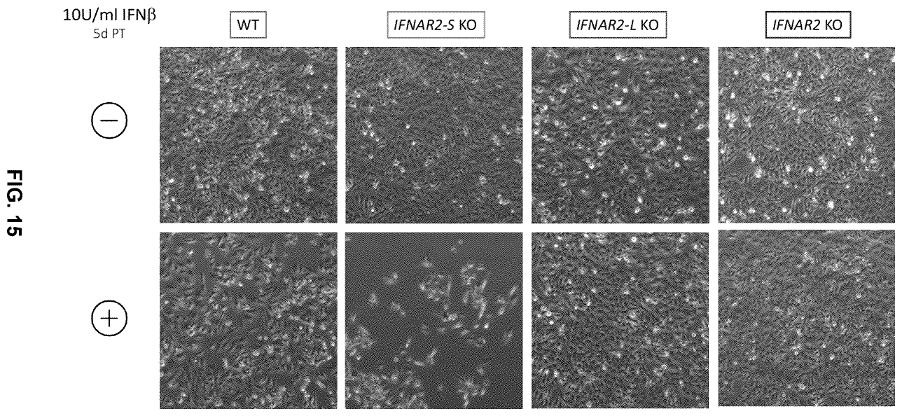

As shown in FIG. 15, specifically that knockout of IFNAR2-L prevents cytotoxic effect of IFNB, and knockout of IFNAR2-S leads to higher cell death and less proliferation by IFNB. Effects of IFN treatment of HeLa with different genomic backgrounds at the IFNAR2 locus. KO mutant Hela cells were grown in 10 U/ml of IFNb for 5 days (bottom row) or with equivalent concentration of PBS (resuspension media of IFNb; top row). After 5 days, pictures were taken using the camera integrated in the microscope at a 4× magnification. Wild type HeLas (WT) respond to IFN with moderate increase in lethality and a slight growth delay. IFNAR2-S KO clones show a more dramatic response to the treatment, whereas IFNAR2-L and IFNAR2 KO cells show no effect of the treatment. The difference between WT and IFNAR2-S KO clones support our hypothesis that IFNAR2-S represents a functional decoy receptor for the type I IFN pathway.

### Example 9: Modulation of IFNAR2-L and IFNAR2-S Transcript Levels in Overexpression Cell Lines in Knockout Backgrounds

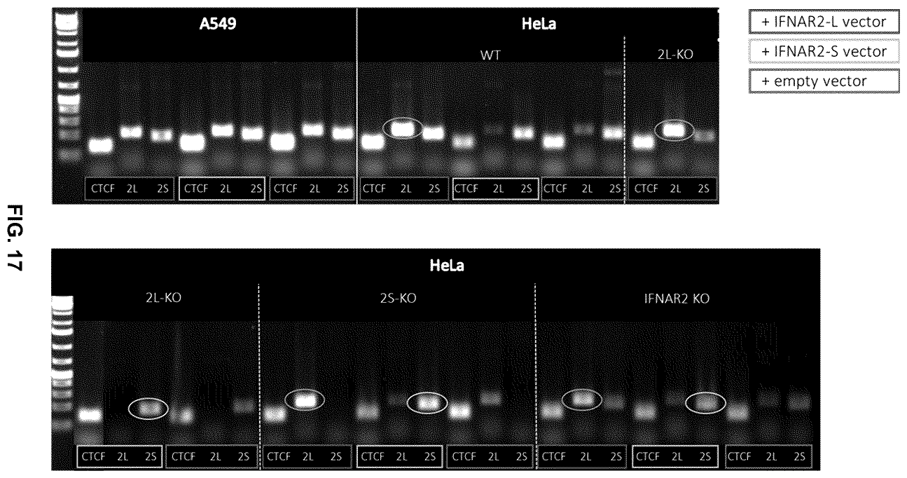

As shown in FIG. 17, preliminary screening of overexpression cell lines. Upon Stable overexpression of IFNAR2-L and IFNAR2-S in multiple cell lines was preliminary assessed by looking at isoform-specific transcript levels. RNA for each cell line was extracted and then reverse-transcribed into cDNA. IFNAR2-L and IFNAR2-S transcript expression was assessed through PCR and electrophoresis using isoform-specific RT-qPCR primers that span the junction between exon 8 (shared exon) and the Alu-Jr derived exon (IFNAR2-S fw) or exon 9 (IFNAR2-L fw). In A549 cells, similar physiological expression levels of the two isoforms makes this approach less valuable. However, in HeLa cells IFNAR2-S is physiologically more expressed than IFNAR2-L, and this allowed for a more straightforward confirmation of our method. Similar confirmation was obtained for overexpression cell lines with a IFNAR2-L KO, IFNAR2-S KO and IFNAR2 KO genetic background. CTCF was used as housekeeping for cross-sample comparisons.

### Example 10: Overexpression of IFNAR2-S Decreases IFN Signaling

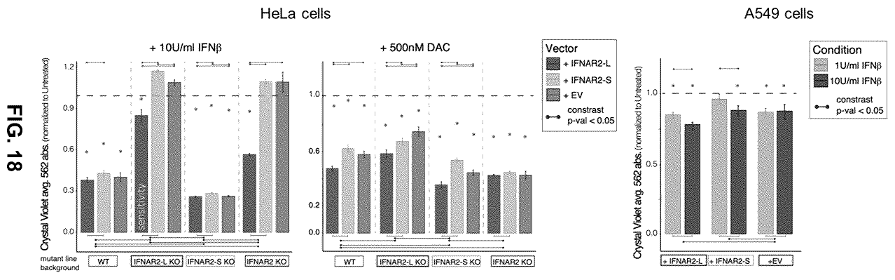

As shown in FIG. 18, overexpression of IFNAR2-L increases cytotoxic effect of IFNB in all KO backgrounds, and overexpression of IFNAR2-S decreases cytotoxic effect. Effects of overexpression of IFNAR2-L and IFNAR2-S on cell proliferation. Histograms show the normalized results of a crystal violet staining assessment of cell density across HeLa and A549 cell lines upon treatment. For the detailed experimental protocol please refer to slide 19 (cell viability assay for HeLa KO cell lines). For HeLa cells, significance of changes in expression levels between treated and untreated cells across overexpression cell lines with the same genomic background is reported on top of the histograms and was assessed performing a pairwise emmeans contrast test in R (contrast(emmeans(model, ˜Construct*Condition), interaction=“pairwise”). The same test (model, ˜Genotype*Construct) was performed to assess whereas the change in cell proliferation (D proliferation) between 2L and 2S overexpression cell lines was significant across lines with different genomic backgrounds (contrasts at the bottom of the histograms). Upon further normalization in respect to the empty vector (not shown), analyses also helped assessing whether we were able to rescue KO cell lines (for instance, D proliferation is not significance between WT and IFNAR2-S KO cells). For A549 we also tested if shifts in relative isoform expression were able to affect cell sensitivity to IFN. For instance, upon overexpression of IFNAR2-L, cells become more sensitive to IFN (* at 1 U/ml). *=Untreated vs. Treated p-val<0.05.

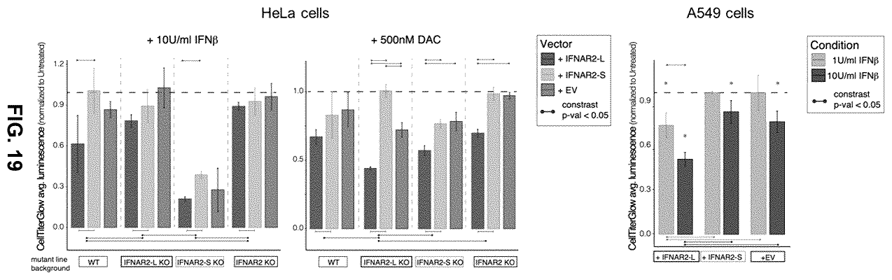

As shown in FIG. 19, overexpression of IFNAR2-L leads to higher cell viability upon treatment, and overexpression of IFNAR2-S leads to higher cell viability upon treatment. Effects of overexpression of IFNAR2-L and IFNAR2-S on cell viability. Histograms show the normalized results of a CellTiter Glo assay of cell viability across HeLa and A549 cell lines upon treatment. Cells were grown in 6 well plates and treated for 3 days in the presence of IFN or for 6 days in the presence of DAC. Cells were then detached and counted. 10,000 cells per each genotype and construct were seeded in triplicate; for the DAC experiment, cells were not treated further. The CellTiter Glo assay, an ATP-base assay for detection of viable cells, was performed the following day. Statistical analyses and data interpretation was performed as for the Crystal Violet assay (FIG. 18).

### Example 11: Isoform-Specific siRNA Recapitulates CRISPR KO

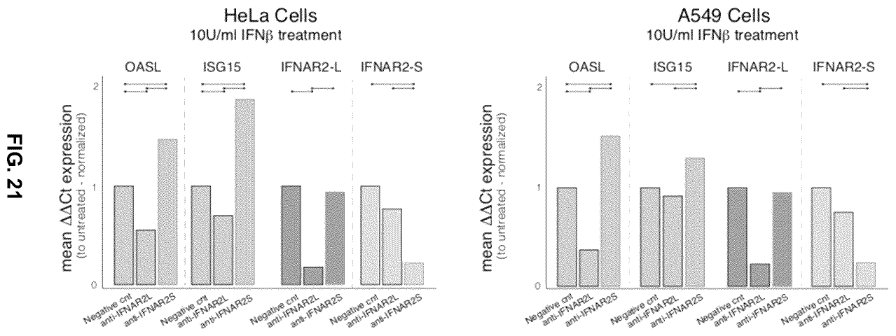

As shown in FIG. 21, isoform specific knock down of IFNAR2-S leads to higher activation of the type-I IFN response. Knock down of IFNAR2-S brings to higher activation of the type I IFN signaling pathway. Histograms show the normalized expression levels of two canonical interferon stimulated genes (ISGs), OASL and ISG15 across HeLa and A549 cell lines as measured by RT-qPCR. Cell were transfected in triplicates with a negative control siRNA, a combo of 2 custom designed siRNAs against the IFNAR2-S isoform (anti-IFNAR2S) and the IFNAR2-L isoform (anti-IFNAR2L). Three days after transfection, cells were treated for 4 hours with 10 U/ml of IFNb and then harvested in lysis buffer. Equal amounts of extracted RNA was used for reverse transcription and quantitative PCR. Normalization was first performed in respect to the Ct of the CTCF gene, here used as housekeeping (DCt), and then in respect to the matched untreated group (DDCt) and in respect to the negative control siRNA. Additionally, we assessed changes in expression levels of IFNAR2-S and IFNAR2-L to validate isoform knock-downs (˜70/80% reduction). In line with our expectations, we observed higher expression of ISGs upon IFN stimulation in cells transfected with the anti-IFNAR2S combo compared to negative control transfected cells. Similarly, we saw a decrease in ISG expression levels when expression of the functional IFNAR2-L is knocked down. We performed a chi-squared test to assess the significance of changes in gene expression levels.

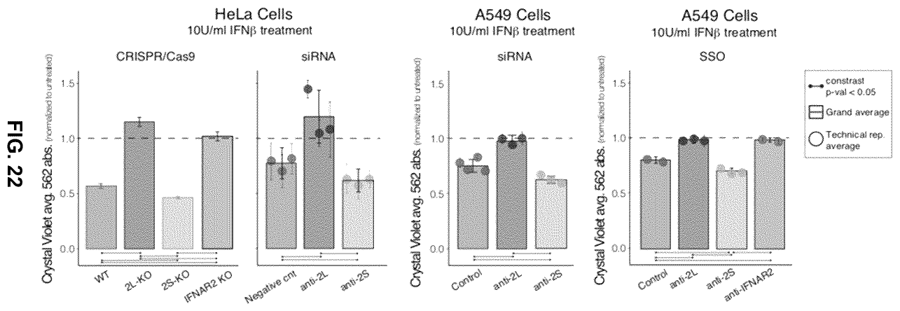

As shown in FIG. 22, isoform specific knock down of IFNAR2-S leads to lower cell proliferation upon treatment. Knock down of IFNAR2-S brings to less proliferation and higher cell death. Histograms show the normalized results of a crystal violet staining assessment of cell density across HeLa and A549 cell lines. An additional plate was prepared for transfection (as in slide 26), but cells were allowed to grow in presence of IFN for additional 3 days. Cells were then passaged with or without IFN at a lower density (1.100 dilution) and subjected to crystal violet staining the following day. For each sample, we averaged absorbance values from 3 technical replicates (dots). Significance of changes in cell density between treated and untreated (*) cells was assessed performing a pairwise t-test. Significance of changes in expression levels between treated and untreated cells across knock down cell lines is reported at the bottom of the histograms and was assessed performing a pairwise emmeans contrast test in R. In line with our expectation, we observed higher expression of ISGs upon IFN stimulation in mutant cell lines that lack the decoy receptor IFNAR2-S (2S-KO, in yellow) compared to wild type cells (WT, in dark grey). We saw no significant changes in ISG expression levels when the functional IFNAR2-L is deleted (2L-KO, in blue). We reported also results from matched experiments using HeLa mutant cells lines (CRISPR/Cas9, on the left) and Sequence-Specific Oligonucleotides (SSOs) designed to block the splicing and processing of the IFNAR2-L (anti-2L, in blue) and the IFNAR2-S (anti-2S, in yellow) isoforms, as well of Exon 7, which is involved in binding to IFN molecules.

### Example 12: IFN Signaling can Depend on Relative Ratio Rather than Raw Abundance of IFNAR2 Isoforms

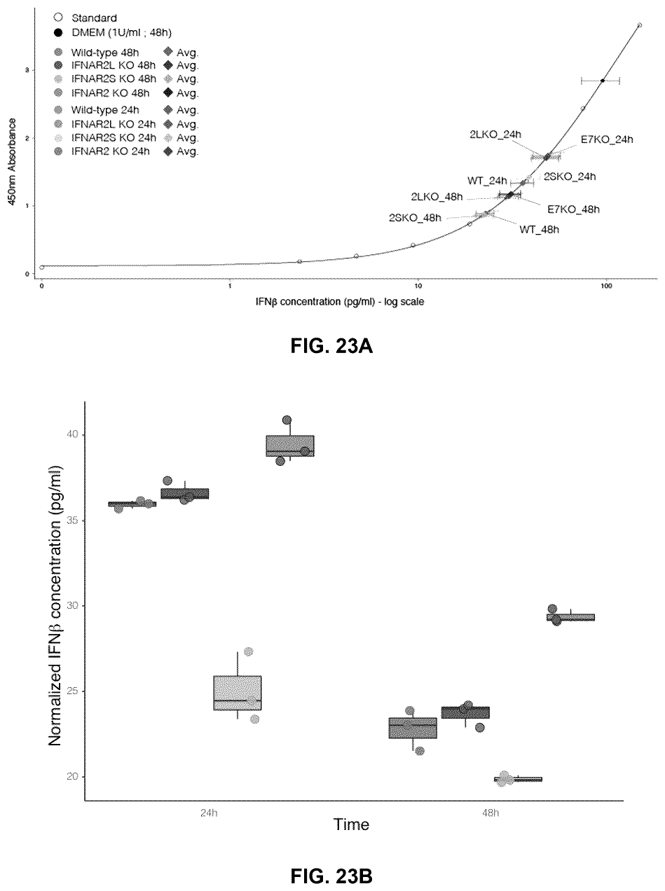

As shown in FIG. 23, IFNAR2-S may act as a transient trap of IFN. (A) Mutant HeLa cell lines were treated with 1 U/ml of IFNb for 24 h and 48 h, when media was collected and subjected to ELISA. On top, the standard curve for IFN concentration shows different levels of IFN internalization across mutant CRISPR/Cas9 KO lines and wild-type HeLa cells that are independent of IFN decay when left for 48 h in incubation at 37 C in absence of cells (black dot). Residual concentration of IFN in the culturing media was calculated by using a 4-parameter fit for the standard curve, correcting for the absorbance of untreated media background. (B) The box plot reflects the inferred residual IFN concentrations in the different cell lines (three biological replicates each) through time. Compared to wild-type HeLas, the IFNAR2 KO cell line shows the lowest levels of IFN internalization. Depletion of IFN from the media compared to culturing media without cells suggest either IFN internalization through IFNAR1, or cellular production and release of compounds that can destabilize IFN molecules. Different patterns between IFNAR-S KO, IFNAR2-L KO and IFNAR2 KO cell lines, on the other hand, does not support internalization of IFN through the IFNAR2-S receptor, although there is support for a transient binding of IFN. Although more mechanistic data is required, this experiment seem to support our claim that the ratio between IFNAR2-L and IFNAR2-S may be critical for cell responsiveness to IFN and the strength of IFN signaling.

1-26. (canceled)

27. A method of modulating a response to type I Interferon IFN) treatment in a subject in need thereof, comprising modulating the relative ratio of type I interferon receptor gene isoform IFNAR2-L and type I interferon receptor gene isoform IFNAR2-S, wherein the level of IFNAR2-L is greater than IFNAR2-R, and wherein the method increases the sensitivity to the INF treatment in the subject.

28. The method of claim 27, wherein said step of increasing the sensitivity to IFN treatment comprises upregulating expression of IFNAR2-L.

29. The method of claim 27, wherein said step of increasing the sensitivity to IFN treatment comprises downregulating expression of IFNAR2-S.

30. The method of claim 27, wherein said step of increasing the sensitivity to IFN treatment comprises knocking out all or part of the gene encoding IFNAR2-S.

31. The method of claim 30, wherein said step of knocking out all or part of the gene encoding IFNAR2-S comprises the step of CRISPR/Cas9-mediated knocking out all or part of the gene encoding IFNAR2-S

32. The method of claim 27, wherein said step of increasing the sensitivity to IFN treatment comprises administering an effective amount of one or more siRNAs targeting the expression of IFNAR2-S.

33. The method of claim 32, wherein said one or more siRNAs comprises one or more siRNAs derived from the sense and antisense sequences selected from SEQ ID NO.'s 1438-1773.

34. The method of claim 27, wherein said step of increasing the sensitivity to IFN treatment comprises administering an effective amount of one or more antisense oligonucleotides (ASOs) targeting the expression of IFNAR2-S.

35. The method of claim 34, wherein said one or more ASOs comprises one or more of ASOs selected from SEQ ID NO.'s 3201-3533.

36. The method of claim 27, wherein said step of increasing the sensitivity to IFN treatment comprises administering an effective amount of one or more splice-switching oligonucleotides (SSOs) configured to mediate preferential splicing of IFNAR2 to generate IFNAR2-L, and wherein said step of preferential splicing comprises preferential splicing of exon 9 of IFNAR2 to preferentially form IFNAR2-L.

37. (canceled)

38. The method of claim 36, wherein said one or more SSOs comprises one or more of SSOs selected from SEQ ID NO.'s 3502, and 3504-3634.

39. (canceled)

40. A method of modulating a response to type I Interferon (IFN) treatment in a subject in need thereof, comprising modulating the relative ratio of type I interferon receptor gene isoform IFNAR2-L and type I interferon receptor gene isoform IFNAR2-S, wherein the level of IFNAR2-L is less than IFNAR2-R, and wherein the method increases the resistance to the INF treatment in the subject.

41. The method of claim 40, wherein said step of increasing the resistance to IFN comprises downregulating expression of IFNAR2-L.

42. The method of claim 40, wherein said step of increasing the resistance to IFN comprises upregulating expression of IFNAR2-S.

43. The method of claim 40, wherein said step of increasing the resistance to IFN comprises administering an effective amount of one or more siRNAs targeting the expression of IFNAR2-L.

44. The method of claim 43, wherein said one or more siRNAs comprises one or more siRNAs derived from the sense and antisense sequences selected from SEQ ID NO.'s 8-1437.

45. The method of claim 40, wherein said step of increasing the resistance to IFN comprises administering an effective amount of one or more antisense oligonucleotides (ASOs) targeting the expression of IFNAR2-L.

46. The method of claim 45, wherein said one or more ASOs comprises one or more of ASOs selected from SEQ ID NO.'s 1774-3200.

47. The method of claim 40, wherein said step of increasing the resistance to IFN comprises administering an effective amount of one or more splice-switching oligonucleotides (SSOs) configured to mediate preferential splicing of IFNAR2 to generate IFNAR2-S, and wherein said step of preferential splicing further comprises preferential splicing of exon 9 of IFNAR2 to preferentially form IFNAR2-S.

48. (canceled)

49. The method of claim 47, wherein said one or more SSOs comprises one or more of SSOs selected from SEQ ID NO.'s 3149-3171, and 3534-3604.

50-61. (canceled)
